SEQUENCE LISTING (32 sequences)

SEQ ID NO: 1 - DNA, 1806 bp, unknown
  mlh1 cDNA sequence from tomato (Lemlh1)
atggaagacgaagccattccagtgccgattccgaaggagccaccgaagattcagcggctggaagaatgtgtggtgaacagaatagcggctggcgaagtcatccaaaggccagtctctgccgtgaaagagctcattgagaacagcctggatgctgattccacctctatttccgttgttgttaaggatggcggtcttaaacttatccaagtttccgacgatggccatggaatccgttatgaagatttgccaattttatgcgagaggtatactacgtccaagctgagtaaatttgaagatttgcagtccattaggtcgatgggatttagaggagaagccttggctagcatgacatatgtgggtcacgtcactgtcaccaccattactatgggccagttgcatggatacagggcaacatatagagatggtttgatggtggatgagccaaaggcttgtgctgctgtcaagggtacccagataatgattgaaaatttattttataacatggctgcacgaaggaaaacccttcaaaattctgccgatgactatccaaaactagtggatcccccgggctgcggaattcatcacacacatgtgagcttctcttgtagaaagcatggagctggtagagcagatgttcacactattgctacttcttcaaggctcgatgcaattagatccgtttatggagcttcagttgctcgagatctgatgaatatcgaagtttctgatactggtccattaatttcagtttttaagatggatggtttcatctccaactctaattatattgcgaagaagacaacaatggtgctttttataaatgatagactcattgattgtggtgctttgaagagggcaattgaaatagtctatactgcaacattgcctaaagcatcaaaacctttcatatacatgtcaatcattttgccgcccgagcatgttgatgtgaatatacacccaacaaagagagaggtaagctttttgaatcaagagttcgtcattgagaagatccagtctgtagtagggtcaaaattgagaagctccaatgagtcgaggacattccaggaacagactatggatttatcttcatctggtccaatgggccaagattccactaaagaatcgtctccttctgggataaagtcacaaaaagtgccacataaaatggtacgaacagatactttggacccttctggaaggctgcacgcttacatgcaaatgaagcctcctggtaattcagaaagaggtccttgctttagctctgtgaggtcttctatcagacaaaggaggaatcctagtgacaccgcagacctcactagcatccaagagctcgttaatgagattgataatgactgtcaccctggtctattggatattgttaggaattgcacatatactgggatggcggatgagatttttgctttgcttcaacacaatacacacctttatcttgttaatgtgattaacttgagtaaagagcttatgtatcagcaagttttacgtcggtttgcccatttcaatgcaattcaactgagtgaaccagcatcattacctgagttagtaatgcttgctctgaaagaagagggttcagatccagaaggcaacgaaagcaaagagctaagaggaaagattgccgagattgaatacagaactgctcaagcaaaaggctggaatgctagaaggagtattttagtattcattatcgattcaaatggaaatatgtctagtcttcctgtatactgggatcagtacacacctgacatgggaccgcatcccagaaatttattactttggttcaggaaatga

SEQ ID NO: 2 - DNA, 1806 bp, unknown
  optimized LeMLH1 encoding nucleic acid sequence
atggaagatgaggctattccagtgccaattccaaaggagccaccaaagattcagaggcttgaggagtgcgttgtgaacaggattgctgctggagaagttattcagaggccagtgtctgctgtgaaggagttgattgagaactctttggatgctgattctacttctatttctgtggtggtgaaggatggaggattgaagttgattcaggtgtcagatgatggacatggaattagatacgaggatttgccaattttgtgcgagagatacactacttctaagttgtctaagttcgaggatcttcagtctattaggagtatgggattcaggggagaggctttggcttctatgacttatgtgggacatgtgactgtgactactattactatgggacagttgcatggatacagggctacttacagggatggattgatggtggatgagccaaaggcttgcgcagctgtgaagggaactcagattatgattgagaatttgttctacaacatggctgctaggagaaagactcttcagaactctgctgatgattacccaaagttggtggatccaccaggatgcggtattcatcatactcatgtgtctttctcttgcaggaaacatggagctggaagggctgatgtgcatacaattgctacttcatctaggcttgatgcaattaggagtgtgtacggagcttctgtggctagggatttgatgaacattgaggtttcagatactggaccattgatttctgtgttcaagatggatggattcatttctaactctaactacattgctaaaaagactactatggtgttgttcattaacgataggcttattgattgcggagctttgaagagggctattgagattgtgtacactgctactcttccaaaggcttctaagccattcatttacatgtctattattcttccaccagagcatgtggatgtgaacattcatccaactaagagggaggtgtcattcttgaaccaggagttcgtgattgagaagattcagtctgtggtgggttctaagttgaggtcatctaacgagtctaggactttccaagagcagactatggatctttcttcttctggaccaatgggacaggattctactaaggagtcatctccatctggtattaagtctcagaaagtgccacataagatggtgaggactgatactttggatccatctggaaggttgcatgcttacatgcaaatgaagccaccaggaaactctgagaggggaccatgcttctcttcagtgaggtcatcaattaggcagaggaggaacccatctgatactgctgatttgacttcaattcaggagcttgtgaacgagattgataacgattgccatccaggattgcttgatattgtgaggaactgcacttacactggaatggcagatgagattttcgctttgcttcagcataacactcatctttaccttgttaacgtgattaacttgtctaaggaattgatgtaccagcaggtgttgaggagattcgctcatttcaacgctattcagttgtctgagccagcttctttgccagagttggtgatgttggctttgaaggaggagggatctgatccagagggaaacgagtctaaggagttgaggggaaagattgctgagattgagtacaggactgctcaggctaagggatggaacgctaggaggagtattcttgtgttcattattgattctaacggaaacatgtcatctttgccagtgtactgggatcagtacactccagatatgggaccacatccaaggaacttgttgttgtggttcaggaagtga

SEQ ID NO: 3 - protein, 601 aa, unknown
  MLH1 protein from tomato (LeMLH1)
MEDEAIPVPIPKEPPKIQRLEECVVNRIAAGEVIQRPVSAVKELIENSLDADSTSISVVVKDGGLKLIQVSDDGHGIRYEDLPILCERYTTSKLSKFEDLQSIRSMGFRGEALASMTYVGHVTVTTITMGQLHGYRATYRDGLMVDEPKACAAVKGTQIMIENLFYNMAARRKTLQNSADDYPKLVDPPGCGIHHTHVSFSCRKHGAGRADVHTIATSSRLDAIRSVYGASVARDLMNIEVSDTGPLISVFKMDGFISNSNYIAKKTTMVLFINDRLIDCGALKRAIEIVYTATLPKASKPFIYMSIILPPEHVDVNIHPTKREVSFLNQEFVIEKIQSVVGSKLRSSNESRTFQEQTMDLSSSGPMGQDSTKESSPSGIKSQKVPHKMVRTDTLDPSGRLHAYMQMKPPGNSERGPCFSSVRSSIRQRRNPSDTADLTSIQELVNEIDNDCHPGLLDIVRNCTYTGMADEIFALLQHNTHLYLVNVINLSKELMYQQVLRRFAHFNAIQLSEPASLPELVMLALKEEGSDPEGNESKELRGKIAEIEYRTAQAKGWNARRSILVFIIDSNGNMSSLPVYWDQYTPDMGPHPRNLLLWFRK

SEQ ID NO: 4 - protein, 195 aa, unknown
  amino acid sequence containing LeMLH1 C-terminal part and used to raise anti-LeMLH1 antibodies
MGSSHHHHHHSSGLVPRGSHMASMTGGQQMGRGSEFELVNEIDNDCHPGLLDIVRNCTYTGMADEIFALLQHNTHLYLVNVINLSKELMYQQVLRRFAHFNAIQLSEPASLPELVMLALKEEGSDPEGNESKELRGKIAEIEYRTAQAKGWNARRSILVFIIDSNGNMSSLPVYWDQYTPDMGPHPRNLLLWFRK

SEQ ID NO: 5 - DNA, 588 bp, unknown
  DNA sequence encoding SEQ ID NO 4
atgggcagcagccatcatcatcatcatcacagcagcggcctggtgccgcgcggcagccatatggctagcatgactggtggacagcaaatgggtcgcggatccgaattcgagctcgttaatgagattgataatgactgtcaccctggtctattggatattgttaggaattgcacatatactgggatggcggatgagatttttgctttgcttcaacacaatacacacctttatcttgttaatgtgattaacttgagtaaagagcttatgtatcagcaagttttacgtcggtttgcccatttcaatgcaattcaactgagtgaaccagcatcattacctgagttagtaatgcttgctctgaaagaagagggttcagatccagaaggcaacgaaagcaaagagctaagaggaaagattgccgagattgaatacagaactgctcaagcaaaaggctggaatgctagaaggagtattttagtattcattatcgattcaaatggaaatatgtctagtcttcctgtatactgggatcagtacacacctgacatgggaccgcatcccagaaatttattactttggttcaggaaatga

SEQ ID NO: 6 - protein, 318 aa, unknown
  amino acid sequence used to raise anti-LeSMC1 antibodies
MGSSHHHHHHSSGLVPRGSHMASMTGGQQMGRDPNSSSVDKLAAALENFKSYKGFQTIGPFYDFTAIIGPNGAGKSNLMDAISFVLGVRTGQLRGAQLKDLIYAFDDREKEQRGRRAFVRLIYQLANGTEIQFTRAITSAGASEYRIDGKAVNWDEYNAKLKSLDILVKARNFLVFQGDVESIASKNPKELSALLEQISGSEEFKRRYDELEEEKARAEEKKALAYQKKKTVTMERKQKKEQKEEAEKHLRLQDKLKSLKQEYFLWQLFNIEKDIAKTNEELDAEEARVKEIVEKLGEYESESSKKKKKKLEHHHHHH

SEQ ID NO: 7 - DNA, 954 bp, unknown
  DNA encoding SEQ ID NO 6
atgggcagcagccatcatcatcatcatcacagcagcggcctggtgccgcgcggcagccatatggctagcatgactggtggacagcaaatgggtcgggatccgaattcgagctccgtcgacaagcttgcggccgcactcgagaatttcaagtcctacaaaggatttcaaacaattggcccattttacgactttacagcgattatagggccaaatggagcaggaaaatctaacctgatggatgcaatcagcttcgtccttggagttcgtacgggtcaacttcgaggtgcacagttgaaggacttaatttatgcttttgacgaccgtgaaaaggagcagagaggtcgaagagctttcgtgaggctaatttatcagcttgctaatggtacggaaattcagtttacgcgtgctattacgagtgctggtgcgagtgagtatcggattgatgggaaagctgttaattgggatgagtataatgccaaattgaagtctcttgatattctggttaaagcacggaattttctcgtctttcagggtgacgttgagtctattgcatctaaaaatccaaaagaactctctgcactccttgagcaaatatctggatctgaagagttcaagagacgctatgatgaattggaagaagaaaaagcaagagctgaagaaaagaaggcacttgcttaccagaaaaagaaaactgtaactatggagcgaaaacagaaaaaggaacagaaagaagaagctgagaagcatcttcgtttacaagataaactgaaatctttgaagcaagaatattttctgtggcaattattcaacatagaaaaggatattgctaagacaaacgaggaacttgatgctgaagaagcaagagtcaaagaaattgtggagaaacttggggaatatgaaagtgaatctagcaaaaaaaaaaaaaaaaaactcgagcaccaccaccaccaccac

SEQ ID NO: 8 - protein, 209 aa, unknown
  amino acid sequence used to raise antiLeCENP-C antibodies
MGSSHHHHHHSSGLVPRGSHMASMTGGQQMGRGSEFGTRAVEDFGSTEIDPLVDNMLPETAPSAEQDHYFEDSVKDLNSDQLNSVGVEVPSRDVRPKFPEMSPQHHKQAKDKQQKAKELAVGRRERKHLSSRPSLADAGTSFESGVRRSKRMKTRPLEYWKGERLLYGRVDEGLKLVGLKYISPGKGSFKVKSYIPDDYKDLVDLAARY

SEQ ID NO: 9 - DNA, 630 bp, unknown
  DNA encoding SEQ ID NO 8
atgggcagcagccatcatcatcatcatcacagcagcggcctggtgccgcgcggcagccatatggctagcatgactggtggacagcaaatgggtcgcggatccgaattcggcacgagagctgttgaggattttggatcaactgaaattgatccgctggttgacaatatgctaccagaaacagcgccttctgcagaacaagatcattactttgaggattctgtcaaagacttaaatagtgatcaattgaactcagtgggagttgaagttccttctagagacgtgagacctaaatttccggagatgagccctcagcatcacaagcaggcaaaagataagcaacagaaagcaaaggaacttgctgttggaaggcgtgaaagaaaacatctttcttctaggccaagtctagcagatgctggcacatcatttgaaagtggggttagacgaagcaaacgaatgaagacaaggcctttggaatattggaaaggcgaaagattattatatggacgggttgatgagggtttgaagcttgttggcttgaaatacatctctccgggaaagggatcatttaaggtgaagtcttacattcctgatgactacaaagacctagttgatttggcagctcgctattga

SEQ ID NO: 10 - protein, 318 aa, unknown
  amino acid sequence used to raise anti-LeSMC3 antibodies
MGSSHHHHHHSSGLVPRGSHMASMTGGQQMGRDPNSSVDVDMLQAEVESKYQELKDADSLVDHVTKELTRVSRNIDERNKRLKQIKQEKDNLKALEDKYQNTLQDEARELEQMLSKRNTYLAKQEDYSKKIRELGPLSSDAFETYKRKNVKELYKMLHKCNEQLQQFSHVIKRHLINMXTLLSKEKNLQEGKLNWMLGMRKSRNLYSVLEDGEEDDNDPDDDEPRADAEGRVEKYIGVKVKVSFTGQGETQPMKQLSGGQKTVVALALIFAIQRCDPAPFYLFDEIDAALDPQYRTAVGNMVRDLADRGSTQFITTTF

SEQ ID NO: 11 - DNA, 954 bp, unknown
  nucleic acid sequence encosing SEQ ID NO 10
atgggcagcagccatcatcatcatcatcacagcagcggcctggtgccgcgcggcagccatatggctagcatgactggtggacagcaaatgggtcgggatccgaattcttctgtcgacgttgatatgctacaggctgaagttgagagcaagtatcaggaactcaaggatgcggactcactagttgatcatgtcaccaaagagcttacaagggtttctcgaaatatagatgaacgaaacaagaggctcaagcagatcaaacaggaaaaggataacctgaaggctcttgaagataagtatcagaatacacttcaggatgaagcaagagaactggaacagatgctaagtaaacggaacacctatcttgcaaagcaagaagattactctaagaaaattcgggaactgggtcctttatcttctgatgcctttgagacgtacaaaagaaagaacgtaaaagaattatataagatgctgcacaagtgtaacgagcagctgcaacagttcagccatgtaataaaaaggcacttgatcaatatgtaaactttactgagcaaagagaagaacctccaagaaggcaagctgaactggatgctggggatgagaaaatcaaggaacttatattctgttttggaggatggtgaggaagatgacaatgatcctgatgatgatgagccgcgtgctgatgcagagggaagagttgagaaatatattggtgtgaaagtgaaggtgtcgttcactggacaaggagaaacacagccaatgaaacagttatcagggggccagaaaactgtggtagcactggcacttatatttgccatacaaagatgtgatcctgccccattttatctttttgatgagatagatgcagcactagatcctcagtacagaacagccgtggggaatatggttcgtgatttggcagacaggggtagcacgcaatttatcacgacgacattt

SEQ ID NO: 12 - DNA, 5062 bp, unknown
  At DMC1 promoter
ggcgcgccagatcaacaccgtttatatgagacaaaatcagctatgagattactcgtgtatcaattctctaattaattaaaaatagtataaattaaataatatagttcgatacacgaatataattgcgaagaataggcatacaaatttgtcatacatgtttcgatatggctcacgaggaggctgatgtaacagtttgatgtatacgtatgcaaattgagaagtacttgatcagacctatatatgtgatgctcgaacttatctttttgttttggatcatctatcgaatacaatggtactataatttaaatgttttttttcttctttttctttagtatcaaaagcaacgttagatgctaaataaagagttagttgattgtgatgactgatagtctgataaaatcattaactttgcacccgaagtcaaataaaagtgttcatatttataaattccaaccaacgttaataagccacacctaatcggtgattgccaacaatattataataaaattaaaaaaactacgactaaagttaatttgctataattttgtggtatgttttaaaaacaaagttctttagttctaatatcatgaaaattcagtgtactgtaaaatatgtaaaaaggttttagtacagttcttttttgtatataacggcaaagttcaatacatattttactattgaattttttttataaaaaaataacaattgctaccaactttttgaagcatattgatcgcaacttaattagaattcttctttttttttcttggaagattaataaaacctaatttcaatgtggaacaaataaatgtagaaatattgttatcacaaactaatatatgatattttttaatattttcatatatacttttgagattctgatgatataacagttttcattaaaatacaaattgtcgtgtactaatttttcttttgttcaagtatgtgataaaaatatgttgcaaattgcgagttattataatgttacaaatatgtagagagaatacatgagaagagttaaaagaagcatgcttaagccaacagagagtggatccaaatgttgctttccagctttatacaaacgtatcacccacattactgccactgctacatatattgaaggagagagagatgatgatcataatgataaatcgatgtcgatgataaattgatgatgatggctccggtatgtgtaccctaggagttgtagctagctagctaggaccatgtatatacatacatacatatattagtgttttttgtaacttgtacgtaccttacatagagtatggagtttactaaaacggcaacgtttggtgggggtagtgaattcgcaagtggggatgagtctatgtaatagaagatgcaacatgcaaatggtcccctttctgtttttatttaaagaaatgagtgtttactgaggaggaaacatcccatttatagattcacacccataaaagcaaaccacttctccttctttttatttccccatgatattacttcgagaatattttgaaaatttgaagtgtacatttagagattgtgtactttgaacactcatgtcaaatgcatctaaatacataaactccaatttaaaataatcgtctaaacctagagtgccatttgtttagccatttgttggtcttcatttctcatgctttgattacatgtaccggttgattcatgtgaaaaatcatgtgcataaactaagaaatagctagcacataaaattttgatttaggttggatattactatgttcactttaagagaaaaaaaacttatggcaaaaagtgatgatggtatatgaatatgataatcaaagtgcatatgtgaagtgagaggcaactgtagagtaatataataaaatccaaagaaaatttttaaatatgagaaaaaattatataaaaaggttcttttgtaatccacttcttttgatatagggagattcgttgagcatccatgtgctctttcaatcgacactattctgtctgtatctagccaaccacatatacctttacactagagaacttcgatgattctttttcaaaatcaatgtgatataatataattaagcatatatgcataaaaaatgaagaagaatggtagagtcatgttacttaaggtcatggtgtgtaaaaacattgatactttacaatatatgagttgtgaagtgctcttaaagttataacatccggttctacgtattgacctagaactagaagaatcgttttttagtccaaatcaaatcaagtcggttctttatcagttttgttgtatgtgaattaatttgaaaatattagctatgatcttagcttgggtttttgtttctaagggttaaggatcatatctctttgtcaaatgacatgtggtctatatgtcatgaattaggcaccgctatcttttactattgattcgacgacattgggactcctcactacacttatcttaaaaaaactcaaagttggtgttaatggcttgtcaccataaactttcatgagctctaacaaattaaacttgaacttgatcaggtctcacaatatatacaatttcgagggataaatatttcaaaaggataatatgatagttggtagaaatgtatagtttctagtaataatagagatcgttggttaaactccccaactttttaaaattaatttgattagtggatccgcaaacaaatattagattgggcctatatgcatctatattatttttatttttctgtaatttcagtaaaatgggcctatggtcctatatgcatccgaataattagtatactgggcttatgggcctatatgcatttgattttatcgataaaatgtgagtcaaatgtctaatgtgcgccgttatgaagtgcaagtggctaatttttttcagggaatgttccaatataagacactttaaacgtaagtttagacaatatagacactttccaagttagaggcacttttccttctttttgaaggaaaacttgacttttatacctcttaactaaacaatcgaaaacaataactaaatatatatcttaaccaaacaattaaaaaaataaaagaatttagatacgtagttattaatatagaccattagattgaaaaataaaaattaagatctatggctgagattaaagacaataaatggattaattttttgatgttaaaatctgattagaaaaaggtatttctcttcgtctctagaactaaatctctctctctaaaaaaacaatcgtttctccctttctccttcctgaagatcgttttttcataaatccatagtagtttaaaaacgaagcagagagatgttgaaaatcgtttctcatgaaattaatcgattattctctgtgaagttctttaatccacacaactttcctcatgaacatgataatagtagtaaatggaggtttttcctatggttactctagacgaaggaggatctccttgtgttggacaggtttgtgatttctttccatggattaaaaaaatttgattgtttgtttatgatgaacgattctttggctacggaagagtgtcatggagttctggcgaattctttggctatgtttggtgatttcgtttttaatcaagttgggaatcaataggaaacaactaagcatacaacatagattagaagagatatcaagatggatctaatttaagtaagatttggcgactaattctagatgattagggttatttgtgatttattacaaggcatttgtgttctcattgatttggcgagtaattctgtatgactagggttatttgtgttttcttaaaaagaatttgtgttcttgttgaaatcttgttcattggaattatttgtgtttggtaaatcttcattggtggctaaggatgtgtttgtagctcttacggcgtttgttattggtgatgtccattatggatggcaaattatggatggcacattatggatgatgaatcatggatgacatattatggatgacgcatcatggattgtatattatggattgatatggtgagatttgtaaatcttttggtcttacatgttaagagtaaaagatgaagaattggagaagcatgtctaacatcctaaaaacaagctatatgcggttgatttgctacaaataattttttggtatccataataacaaatccatttaaatatatccattcagaaacctttctactgatccgtatccattctatataccatgtcaataataataggagattcgattaaccgtgttttgtaaagaaaccaaagttccatgtccataaggttttgaaggtggaggtctctgcaaactgaaaaaaaaatcaacaaacaattttttggtgtccataataacgaatccatttaaatatatccattcggaaacctttttactgatctatatccattttatataacatgtccatgataacaggagattcgattaactgaaatctcgatgctacgtagatgaaacgagtttgacacatgagagagagcaaaaatcaaatcaaaccgccattgttgaagaagaagaagtttcttctcattttttacaaagatgaagagagagagaggtgaagagagagagagagatgaagagagagagagagagaaagagagagatgaagagagagagagaaagagagaaaacgtgggttaagataatattttagttaagagggtattttagtaaaaaaacataaaaaagtgcctaatcttttgaaagtgcctaaacacagaaatagttttaaaaaagtgtttaagagtgtaatattctctttttttcacctagattccttctattgaccgtcgatagacggatgataactatgacgtggcattatcgcagccatcaaacaaagtcatgtataacaaagaagagcacacaaacgaaaacaaattcagttgcggaacccaaattcaaatcaacggaattagaatcacgctttcaattccgtaacccgccattaaaaaccttgaaccctcgaagcaaatcgagcaaagattttcaaatttcgaatttcaaaattctatctctctcactcttccaagcttagagactcttagagcgagaaaagcggccgcccatgg

SEQ ID NO: 13 - protein, 737 aa, unknown
  Arabidopsis MLH1
MIDDSSLTAEMEEEESPATTIVPREPPKIQRLEESVVNRIAAGEVIQRPVSAVKELVENSLDADSSSISVVVKDGGLKLIQVSDDGHGIRREDLPILCERHTTSKLTKFEDLFSLSSMGFRGEALASMTYVAHVTVTTITKGQIHGYRVSYRDGVMEHEPKACAAVKGTQIMVENLFYNMIARRKTLQNSADDYGKIVDLLSRMAIHYNNVSFSCRKHGAVKADVHSVVSPSRLDSIRSVYGVSVAKNLMKVEVSSCDSSGCTFDMEGFISNSNYVAKKTILVLFINDRLVECSALKRAIEIVYAATLPKASKPFVYMSINLPREHVDINIHPTKKEVSLLNQEIIIEMIQSEVEVKLRNANDTRTFQEQKVEYIQSTLTSQKSDSPVSQKPSGQKTQKVPVNKMVRTDSSDPAGRLHAFLQPKPQSLPDKVSSLSVVRSSVRQRRNPKETADLSSVQELIAGVDSCCHPGMLETVRNCTYVGMADDVFALVQYNTHLYLANVVNLSKELMYQQTLRRFAHFNAIQLSDPAPLSELILLALKEEDLDPGNDTKDDLKERIAEMNTELLKEKAEMLEEYFSVHIDSSANLSRLPVILDQYTPDMDRVPEFLLCLGNDVEWEDEKSCFQGVSAAIGNFYAMHPPLLPNPSGDGIQFYSKRGESSQEKSDLEGNVDMEDNLDQDLLSDAENAWAQREWSIQHVLFPSMRLFLKPPASMASNGTFVKVASLEKLYKIFERC

SEQ ID NO: 14 - protein, 707 aa, unknown
  Rice MLH1
IRRLEESVVNRIAAGEVIQRPSSAVKELIENSLDAGASSVSVAVKDGGLKLIQVSDDGHGIRFEDLAILCERHTTSKLSAYEDLQTIKSMGFRGEALASMTYVGHVTVTTITEGQLHGYRVSYRDGVMENEPKPCAAVKGTQVMVENLFYNMVARKKTLQNSNDDYPKIVDFISRFAVHHINVTFSCRKHGANRADVHSASTSSRLDAIRSVYGASVVRDLIEIKVSYEDAADSIFKMDGYISNANYVAKKITMILFINDRLVDCTALKRAIEFVYSATLPQASKPFIYMSIHLPSEHVDVNIHPTKKEVSLLNQERIIETIRNAIEEKLMNSNTTRIFQTQALNLSGIAQANPQKDKVSEASMGSGTKSQKIPVSQMVRTDPRNPSGRLHTYWHGQSSNLEKKFDLVSVRNVVRSRRNQKDAGDLSSRHELLVEIDSSFHPGLLDIVKNCTYVGLADEAFALIQHNTRLYLVNVVNISKELMYQQALCRFGNFNAIQLSEPAPLQELLVMALKDDELMSDEKDDEKLEIAEVNTEILKENAEMINEYFSIHIDQDGKLTRLPVVLDQYTPDMDRLPEFVLALGNDVTWDDEKECFRTVASAVGNFYALHPPILPNPSGNGIHLYKKNRDSMADEHAENDLISDENDVDQELLAEAEAAWAQREWTIQHVLFPSMRLFLKPPKSMATDGTFVQVASLEKLYKIFERC

SEQ ID NO: 15 - protein, 189 aa, unknown
  Allium MLH1 fragment
IEDVTAVDELVRDIDQNVHPGLLDIVKNCTYVGLADETFALLQYNTHLYLMNVINVSKELMYQQAISKFGKFNAIQLSNPAPLQELLIMALKDDNLDPSDADDDELKNKIAEMNTELLKKKAELLDVCYGIWIDDNGNLTRLPVVLEQYTPDMDYVPEFCLSLGNDVDWDDETGCYQTIAATIANFYAM

SEQ ID NO: 16 - protein, 222 aa, unknown
  Allium MLH1 fragment
IRRLDPTVVNRIAAGEVVQRPSSAVKELIENSLDAGATTISVAVKDGGLKLIVVSDNGHGIRFEDLPILCERHTTSKLSKYEDLQMIKSMGFRGEALSSMTYVGHVTVTTITEGQVHGYRVSYKDGMMEDDPEPCAAVKGTQIWVENLFYNMIARRKAFQNCNDEYPKVVDLISRFAIHNINVSFTCRKHGTVRADVHTGNARTRLDAIKTVYGISVARDLM

SEQ ID NO: 17 - protein, 64 aa, unknown
  Cotton MLH1 fragment
IRRLEESVVNHIAAGEVIHRPSSAVKELVENNIDADSSTISVTVKDGGLKLIQVSGGGHGIRME

SEQ ID NO: 18 - protein, 161 aa, unknown
  Soybean MLH1 fragment
IQRLSESVVNRIAAGEVIQRPVSAVKELVENSLDAASSSVSLLIKDGGLKLIQVSDDGHGIRFEDLPILCERHTTSKLSSFEDLQRIKSMGFRGEALASMTYVAHVTVTTITKPQLHGYRVSYRDGVMEHQPRPCAAVKGTQIMVENLFYNMAARRKTLQN

SEQ ID NO: 19 - protein, 204 aa, unknown
  Zea mays MLH1 fragment
PAPLQELLLMALKDDESIXDENDEEKLEIAEANSKILKENAEMINEYFSIHVDPDGNLTRLPVVLDQYTPDMDRLPEFVLTMGNDVTWDDEKECFRTAAAAIGNFYALHPPILPNPSGSGVQVYKKNKACMASGEHVDSTDEDDIDHELLAEAETAWSQREWTIQHVLFPSMRLFLKPPKSMATDGTFVQIASLEKLYRIFERC

SEQ ID NO: 20 - protein, 150 aa, unknown
  Barley MLH1 fragment
DLSSRHELVTEIDSNLHPGLFDIVKNCTYVGVADEVFALIQHNTLLYLVNVVNVSKELMYQQALCRFGNFNAIKLSEPAPLLELLRMALKDDESMSDVNEKEKLEIAEANTEILKENAEMINEYFSIHIDQGGNLTRLPVVLDQYTPDMD

SEQ ID NO: 21 - protein, 188 aa, unknown
  barley MLH1 fragment
DGHGIRCEDLPILCERHTTSKLSAYEDLQTIKSMGFRGEALASMTYVGHVTVTTITEGQLHGYRVSYRDGVMENDPKPCAAVKGTQIMVENLFYNMVARRKTLQNSNDDYPKIVDFISRFAVHHINVNFSCRKHGANRADVHSGSTSSRLDAIRNVYGASVVRDLMEIQVSDENAVDEIFKMDGFISN

SEQ ID NO: 22 - protein, 61 aa, unknown
  Barley MLH1 fragment
IRRLEESVVNRIAAGEVIQRPSSVMKDLVENNIDADSFTISITVKDGGLKLIQVSDGGHGI

SEQ ID NO: 23 - protein, 55 aa, unknown
  Barley MLH1 fragment
EILKENAEMINEYYFSIHIDQGGNLTRLLVVLDQYTPDMDRLPEFMLSLENDVGF

SEQ ID NO: 24 - protein, 151 aa, unknown
  Tomato MLH1 fragment
IQRLEECVVNRIAAGEVIQRPVSAVKELIENSLDADSTSISVVVKDGGLKLIQVSDDGHGIRYEDLPILCERYTTSKLSKGEDFQSIRSMGFRGEALASMTYVGHVTVTTITMGQLHGYRATYKDGLMVDEPKAWAAVKGTHIMIENLFYN

SEQ ID NO: 25 - protein, 170 aa, unknown
  Medicago truncatula MLH1 fragment
IQRLAESVVNRIAAGEVIQRPVSAVKELVENSLDAASTSINLTIKDGGLKLIQVSDDGHGIRREDLPILCERHTTSKLSAFEDLQRITSMGFRGEALASMTYVAHVTVTTITKGQLHGYRVSYRDGVMEQEPRPCAAVKGTQIMVENLFYNMAXRKKTLQNSSDDYSKIV

SEQ ID NO: 26 - protein, 123 aa, unknown
  Phaseolus coccineus MLH1 fragment
LALHSMIQHPVSVVKVLIGNSLDASASTVSVLIKDGGLKLIQVTDNGHGIRVEDLLHIKSMGFRGEALTLMSYFGHVTVTTIFKGQLHGYRVSYRDGVMEDEPMPCAAVGGTQIMVEDLFYNM

SEQ ID NO: 27 - protein, 79 aa, unknown
  Physcomitrella patents MLH1 fragment
IKRLEEAVVNRIAAGEVIQRPASALKELLENSLDAGATSIGITIKDGGLKLIQIVDNGHGIRYEDLPLLCERHATSKLQ

SEQ ID NO: 28 - protein, 172 aa, unknown
  Saccharum officinarum MLH1 fragment
DLSSRHELLMEIDSHCHPGLLEVIKNCTYVGLADEVFALIQHNTHLYLVNVVNVSKELMYQQALCRFGNFNAIQLSEPAPLQELLLMALNDDELIGDENDEEKLEIAEVNLKILKENSEMINEYFSIHVDQDGNLTRLPVVLDQYTPDMDRLPEFVLTMGNDVTWDDEKKCF

SEQ ID NO: 29 - protein, 248 aa, unknown
  Saccharum officinarum MLH1 fragment
DLQTIKSMGFRGEALASMTYVGHVTVTTITEGQLHGYRVCYKDGVMENEPKPCAAVKGTQVMVENLFYNMVARRKTLQNSNDDYPKIVDFISRFAVHHINVNFSCRKHGANRADVHSSSTSSRLDAIRNIYGASVVRDLIEIEVSDEDAGDAVFKMDGYISNANYVAKKIMMILFINDRLVDCTALKRAIEFVYSATLPQASKPFIYMXINLPSKHGDVNIHPTXKKEVAFLXKNRLLETIKNPIEGK

SEQ ID NO: 30 - protein, 333 aa, unknown
  Solanum tuberosum MLH1 fragment
ILRLEECVVNRIAAGEVIQRPVSAVKELIENSLDADSTSISVVVKDGGLKLIQVSDDGHGICYEDLPILCERHTTSKLSKFEDLQSIRSMGFRGEALASMTYVGHVTVTTITMGQLHGYRATYRDGLMVDEPKACAAVKGTQIMIENLFYNMAARRKTLHNSADDYPKIVDLISRFAIHHTHVSFSCRKHGAGRADVHTIATSSRLDAIRSVXYGVSVARNLMNIEVSDTGPLNSVSKMDGFISNSNYIAKKITMVLFIHXRLVDCGALTRAIELVYTATLPKASKPFIYMSIILPPEHVDVNIRANKAERYALLNHEXSSFEKIQSVSRSKL

SEQ ID NO: 31 - protein, 284 aa, unknown
  Triticum aestivum MLH1 fragment
DLSSRHELVTEIDYNLHPGLFDIVKNCTYVGVADEVFALIQHNTLLYLVNVVNVSKELMYQQALCRFGNFNAIQLSEPAPLLELLTMALKDDESMSDVNEKEKLEIAEVNTEILKENAEMINEYFSIHIDQGGNLTRLPVVLDQYTPDMDRLPEFMLTLGNDIAWDVEKECFRTAAAAIGNFYALXSHPSKSIWQRHSIIQEXKDSMESAGQADNDLTSTDEDDIDQELLAEAEAAWAQREWTIQHVLFPSMRLFLKPPKSMATDGTFVQIASLDKLYKIFERC

SEQ ID NO: 32 - protein, 207 aa, unknown
  Triticum aestivum MLH1 fragment
DLPILCERHTTSKLSAYEDLQTIKSMGFRGEALASMTYVGHVTVTTITEGQLHGYRVSYRDGVMENDPKPCAAVKGTQVMVENLFYNMVARRKTLQNSNDDYPKIVDFISRFAVHHINVNFSYRKHGANRADVHSGSTSSRLDAIRNVYGASVVRDLMEIQVSDENAVDEIFKMDGFISNANYVAKKTTMILFINDRLVDCTSLKRA

(19) United States
(12) Patent Application Publication
(10) Pub. No.: US 2009/0031444 A1
(43) Pub. Date: Jan. 29, 2009

(54) HOMOLOGOUS RECOMBINATION IN PLANTS

(76) Inventors: Peter Egbertus Wittich, Blacksburg, VA (US); Frank Georges Paul Lhuissier, Wageningen (NL); Christina Heyting, Bennekom (NL); Hildo Harmen Offenberg, Oss (NL); Ilona Margetha Bruggeman, Wageningen (NL)

Correspondence Address: BROWDY AND NEIMARK, P.L.L.C.;624 NINTH STREET, NW, SUITE 300, WASHINGTON, DC, 20001-5303, US

(21) Appl. No.: 12/065,993

(22) PCT Filed: Sep. 6, 2006
(86) PCT No.: PCT/NL2006/050223

§ 371 Date: Sep. 19, 2008

(30) Foreign Application Priority Data
Sep. 9, 2005 (NL) 2005/000655

(51) Int. Cl. C12N 15/11; C12N 5/02; A01H 5/00; C12Q 1/68; C07K 16/00; C07K 14/00

(52) U.S. Cl. 800/278; 435/468; 800/298; 435/6; 530/387.1; 530/388.1; 530/350; 536/23.1

(57) ABSTRACT

The invention relates to the field of meiotic homologous recombination in plants. Provided are transgenic plants, cytological assays and MLH1 protein and nucleic acid sequences, as well anti-MLH1 antibodies, anti-SMC1, anti-SMC3 and anti-CENP-C antibodies.

### FIELD OF THE INVENTION

The present invention relates to the field of biotechnology, in particular to methods for altering the meiotic homologous recombination frequency and/or the chromosomal location of recombination events in plants and cytological assays for measuring interference-sensitive meiotic homologous recombination frequency and/or the chromosomal location of recombination events. Also provided are novel proteins, and nucleic acid sequences encoding MLH1 proteins, for use in said methods, as well as transgenic plants and plant cells. In a further embodiment antibodies and sequences suitable for raising these are provided.

### BACKGROUND OF THE INVENTION

Plant and animal genomes are commonly characterized by genetic linkage maps, which are maps representing the position of molecular or phenotypic markers along chromosomes or within linkage groups as determined based on recombination frequencies (RF). Such genetic maps are used by breeders in the development of new plant and animal varieties or breeding lines. However, recombination is not evenly distributed along the chromosomes, so that recombination “hot spots” occur, while other regions on the chromosome do not recombine (“cold spots”). For example, crossing over events between homologous chromosomes may be uncommon near the centromeres or in heterochromatin. As a result it is not possible to generate recombinants having an altered genetic make up in those regions or very large numbers of plants are needed to find recombination events in those regions.

A reduced recombination frequency in parts of the genome can, thus, severely hamper breeding progress, as for example undesired alleles positioned at a locus near a desired allele are difficult to remove, resulting in co-inheritance of the chromosomal region. This phenomenon is generally referred to as “linkage drag” or “genetic drag” and is often seen when alleles are introgressed from wild relatives into cultivated species. Also, often very large numbers of progeny need to be generated in order to maximize the chances of finding a recombinant which has the desired characteristics and allele combination. In order to assist in the identification of suitable recombinants, breeders nowadays make routinely use of molecular markers, such as PCR based markers (e.g. AFLP markers, SNP markers, e.g. SNPWave™), which facilitate a more directed screening and enable faster screening of large numbers of progeny, whereby costs can be reduced. Often markers flanking a particular gene of interest are used to detect the presence or absence of the gene.

In addition to the frequency of homologous recombination, especially meiotic homologous recombination, being a limiting factor in the generation of plants, the location and distribution of the recombination events on the chromosomes can be limiting. Methods for increasing the frequency of meiotic homologous recombination and methods for altering the chromosomal location or distribution of recombination events would, therefore, be beneficial in plant breeding, e.g. for removing linkage drag and in reducing the size of breeding populations (and costs associated therewith). Increasing or decreasing meiotic homologous recombination may also be used to increase the genetic variation in a certain crop. Vice versa, methods for decreasing the frequency of homologous recombination would enable the stable maintenance of chromosomes and allelic combinations, which is desired once a good combination of characteristics has been achieved. These approaches for example enable the pyramiding of tandem gene alleles and their stable maintenance. Alternatively, the parental genomes of a cross can be reconstructed, in so-called “reverse-breeding” approaches, see WO03/017753. However, no reliable methods are available for controlling the frequency and position of meiotic crossovers, and it is an object of the present invention to provide such methods.

Homologous recombination is a phenomenon that takes place during meiosis, i.e. the process by which the number of chromosomes per cell is reduced, usually from diploid to haploid, that precedes the formation of (precursors of) gametes. Chromosomes duplicate and enter meiosis with two chromatids (sister chromatids). During meiotic prophase I the chromosomes condense to form long thin threads in leptotene. Each chromosome acquires a proteinaceous axial element to which the two sister chromatids are attached. The homologous chromosomes become aligned during zygotene, forming the socalled synaptonemal complex (SC). At pachytene non-sister chromatids of homologous chromosomes can exchange corresponding parts (recombine), which leads to the formation of chiasmata, and subsequently to the formation of recombinant chromosomes. The two cell divisions that follow lead to the production of (precursors of) gametes, each having a single chromosome set which may, thus, contain recombinant chromosomes. Homologous recombination may also take place during mitosis (in somatic or vegetative cells), though usually at much lower frequencies.

For developing methods for influencing meiotic recombination, efficient assays of both the frequency and the position of meiotic crossovers are required. The frequency and distribution of meiotic homologous recombination is to date measured primarily in genetic studies using markers, such as molecular markers (e.g. AFLPs, RFLPs. Microsatellites, Single Nucleotide Polymorphisms) or phenotypic markers or by using ultrastructural cytological assays using Electron Microscopy, as described by Sherman and Stack, 1995 (Genetics 141: 683-708). Using ultrastructural cytological assays wherein Recombination Nodules (RNs) are physically visualized on chromosome spreads, maps can be generated that show the frequency and position of RNs and can be used to estimate crossing over rates in whole genomes, whole chromosomes or chromosome parts. These methods are laborious and expensive and there is a need for simpler methods. For example, RNs are to date visualized using Electron Microscopy of synaptonemal complexes (SCs) in pachytene bivalents (see Sherman and Stack, supra, and Anderson et al. 2004, Genetics 166, p 1924). Also, genetic linkage maps could be easier combined with pachytene chromosome maps, as described by Andersn et al. (2004; Genetics 166: 1923-1933), if alternative methods to utlrastructural cytological assays were available.

In addition, in a number of eukaryotic organisms evidence has been accumulating for the existence of at least two classes of meiotic crossovers namely interfering and non-interfering crossovers. Interference (also referred to as “crossover interference” or “chiasma interference”) refers to the effect found in eukaryotes that one chromosomal crossing-over event affects the probability that a second crossover takes place in the vicinity of the first. In most organisms the occurrence of one crossover inhibits the occurrence of another in a distance dependent manner, so that crossovers are distributed more evenly along the chromosomes than expected if they were positioned randomly (Jones, 1984, Symp. Soc. Exp. Biol. 38, 293-320). For example, Copenhaver et al. (2002, Genetics 160, 1631-1639), Higgins et al. (2004, Genes Dev. 18, 2557-2570) and Mercier et al. (2005, Current Biology Vol 15, 691-701) provide evidence supporting the hypothesis that two crossover pathways exist in Arabidopsis, only one of which displays interference. Similarly, two cross over pathways appear to exist in humans (Housworth and Stahl, 2003, Am. J. Genet. 73 and Broman and Weber, 2000, Am. J. Hum. Genet. 66:1911-1926) and mice, while in yeast a third pathway (a deleterious cross over pathway) has been suggested (Argueso et al. 2004, Genetics. 168(4):1805-16). Various genes have been speculated to be involved in either of these pathways, mostly genes which are involved in the meiotic recombination machinery and in particular in double-strand break repair. The data is becoming increasingly complex and especially in plants there is still unclarity. In Arabidopsis, for example, allelic mer3 mutants showed a 75% decrease in meiotic crossovers in genetic studies, with the interfering class of crossovers specifically affected (Mercier et al. 2005, supra).

Up to date no cytological method exists for distinguishing between different types of crossing-over (CO) pathways, such as the pathways yielding respectively interfering and non-interfering crossovers, which have been proposed to exist in plants. It was surprisingly found by the present inventors that the use of certain types of antibodies enables discriminating between RNs representing interfering crossovers and RNs representing non-interfering crossovers so that cytological assays could be developed that only measure one specific type of RN, and thus of crossover, or measure the difference in the contribution of the two pathways yielding interfering and non-interfering crossovers to meiotic recombination. In one embodiment this finding makes use of anti-MLH1 antibodies in plants.

MLH1 (mutL homolog 1) is a protein involved in the cellular mismatch repair system and was first isolated from humans (hMLH1, Bronner et al. 1994, Nature, 368, 258-261). WO02/24890 describes a rice ortholog of MLH1 and methods of inhibiting the plant cellular mismatch repair system by either expressing an inactive MLH1 protein or by silencing of the endogenous genes encoding MLH1. The inhibition of the cellular mismatch repair system apparently increases rates of mutagenesis and non-specific recombination events (see page 19, lines 14-17). There is no indication that overexpression of a functional MLH1 protein in a plant, especially using a meiosis specific or meiosis preferred promoter, alters the frequency of meiotic homologous recombination (recombination between homologous chromosome pairs) and/or the distribution and chromosomal location of recombination events. In addition, there is no indication that over-expression is difficult to achieve using wild type mlh1 nucleic acid sequences. The rice ortholog, is only the second MLH1 protein of plant origin and has 66.6% amino acid identity with the Arabidopsis MLH1 protein described by Jean et al. (1999, Mol Gen Genet. 262: 633-343; Accession number AJ012747).

### GENERAL DEFINITIONS

“Recombinant” refers herein to an organism containing one or more chromosomes having a different combination of alleles from either of its parents.

“Recombinant plant” refers, thus, to a plant having one or more chromosomes which have a different combinations of alleles from the parental chromosomes, especially due to crossing over.

“A population of recombinant plants” is herein a population of plants derived from the use of a transformed plant according to the invention, either by selfing and/or crossing said transgenic plant and obtaining the seeds of said selfing and/or cross.

“Crossing over” or “crossover” refers to the reciprocal exchange of chromosome arms and can, for example, be visualized at late stages of meiotic prophase I as chiasmata.

“Homologous recombination” refers to a reciprocal exchange at corresponding positions between homologous chromosomes, such as between non-sister chromatids of homologous chromosomes during meiosis. Homologous recombination can also occur in somatic cells during mitosis (somatic crossing over).

“Meiotic homologous recombination” refers to homologous recombination which takes place between non-sister chromatids of homologous chromosomes during meiosis (in contrast to somatic homologous recombination).

“Homologous chromosomes” are chromosomes within a cell that are identical or very similar in appearance and genetic content, and that pair during the prophase of the first meiotic division (meiotic prophase I). For example, in somatic resp. vegetative cells of diploid organisms (2n) two copies of each chromosome (homologues) exist, one originating from each parent.

“Recombination nodules” (RNs) are submicroscopic spherical or ellipsoidal structures that lie in the central region of the synaptonemal complexes (SC) during mid to late pachynema and are correlated with crossing over and chiasmata (see Anderson et al. 2004, Genetics 166: p 1924, first paragr.). One can distinguish between “early RNs” and “late RNs” based on time of appearance, size, shape and numbers per SC. Late RNs are found at sites of crossing over along synaptonemal complexes (SC) during pachytene. In contrast, early RNs are associated with axial elements and SCs from leptotene through to early pachytene. One late RN represents one crossover event between two homologous, non-sister chromatids, which yields two recombinant and two parental chromatids.

“Gametes” refer to the cells obtained after meiosis, or cells descending from them, that can fuse with another gamete to form a zygote, e.g. the sperm and egg cells of animals and plants. In plants the 4 cells after meiosis develop further. In the anther each cell forms a pollen grain with a (vegetative) nucleus and two sperm cells, in the ovule an embryosac develops consisting of an egg cell, a central cell and often antipodal cells and synergids.

“Recombination frequency” (RF) or “homologous RF” refers to the average number of crossover events per cell/nucleus or per chromosome or per defined subregion of a chromosome. Thus, the RF for a number of cells/nuclei, or of individual chromosomes, needs to be determined.

“Meiotic homologous recombination frequency” refers to the average RF per cell/nucleus during meiosis or per chromosome. It can be determined by calculating the average number of late RNs per cell/nucleus or per chromosome using Electron Microscopy, as described by Sherman and Stack, 1995 (supra). Both interfering and non-interfering crossovers are encompassed herein, because the number of late RNs per cell equals the number of all crossovers per cell. RF can also be determined in a segregating population using genetic markers such as AFLP markers, SNPs or SSRs.

“Frequency of meiotic interfering crossovers” refers to the average number of interfering crossovers per cell/nucleus or per chromosome. It can be determined by determining the number of MLH1-foci per cell/nucleus or per chromosome, using e.g. anti-MLH1 antibodies as described herein.

“Altered recombination frequency” refers to a statistically significant increase or decrease of the average recombination frequency compared to controls.

“Altered frequency of meiotic interfering crossovers” or “altered frequency of interfering meiotic homologous recombination” refers to a statistically significant increase or decrease in the average frequency of meiotic interfering crossovers (see above). “Interfering crossovers” are crossovers that are both interference sensitive and which exert interference.

“Crossover interference” or “interference” refers herein to the nonrandom placement of crossovers along chromosomes in meiosis due to the influence of each interfering crossover on the probability of another crossover in its vicinity.

“Distribution” “location” or “positioning” of meiotic homologous recombination events refers to the physical positions of recombination events on one or more chromosomes of a cell. An “altered distribution” “altered location” or “altered positioning” refers to a relative change in the physical position of the recombination event, without necessarily having an effect on the frequency of recombination.

As used herein, the term “allele(s)” means any of one or more alternative forms of a gene at a particular locus, all of which alleles relate to one trait or characteristic at a specific locus. In a diploid cell of an organism, alleles of a given gene are located at a specific location, or locus (loci plural) on a chromosome. One allele is present on each of the two homologous chromosomes. A diploid plant species may have different alleles at corresponding loci on homologous chromosomes.

“Transgenic plant” or “transformed plant” refers herein to a plant or plant cell having been transformed with a chimeric gene. Said chimeric gene may or may not be integrated into the plant's genome. In a preferred embodiment it is not integrated. A transgenic plant cell may refer to a plant cell in isolation or in tissue culture, or to a plant cell contained in a plant or in a differentiated organ or tissue, and both possibilities are specifically included herein. Hence, a reference to a plant cell in the description or claims is not meant to refer only to isolated cells or protoplasts in culture, but refers to any plant cell, wherever it may be located or in whatever type of plant tissue or organ it may be present.

The term “nucleic acid sequence” (or nucleic acid molecule) refers to a DNA or RNA molecule in single or double stranded form, particularly a DNA molecule encoding a protein or protein fragment according to the invention. An “isolated nucleic acid sequence” refers to a nucleic acid sequence which is no longer in the natural environment from which it was isolated, e.g. the nucleic acid sequence in a bacterial host cell or in the plant nuclear or plastid genome.

The terms “protein” or “polypeptide” are used interchangeably and refer to molecules consisting of a chain of amino acids, without reference to a specific mode of action, size, 3 dimensional structure or origin. A “fragment” or “portion” of a protein may thus still be referred to as a “protein”. An “isolated protein” is used to refer to a protein which is no longer in its natural environment, for example in vitro or in a recombinant bacterial or plant host cell.

The term “gene” means a DNA sequence comprising a region (transcribed region), which is transcribed into an RNA molecule (e.g. an mRNA) in a cell, operably linked to suitable regulatory regions (e.g. a promoter). A gene may thus comprise several operably linked sequences, such as a promoter, a 5′ leader sequence comprising e.g. sequences involved in translation initiation, a (protein) coding region (cDNA or genomic DNA) and a 3′non-translated sequence comprising e.g. transcription termination sites.

A “chimeric gene” (or recombinant gene) refers to any gene, which is not normally found in nature in a species, in particular a gene in which one or more parts of the nucleic acid sequence are present that are not associated with each other in nature. For example the promoter is not associated in nature with part or all of the transcribed region or with another regulatory region. The term “chimeric gene” is understood to include expression constructs in which a promoter or transcription regulatory sequence is operably linked to one or more coding sequences or to an antisense (reverse complement of the sense strand) or inverted repeat sequence (sense and antisense, whereby the RNA transcript forms double stranded RNA upon transcription).

“Expression of a gene” refers to the process wherein a DNA region, which is operably linked to appropriate regulatory regions, particularly a promoter, is transcribed into an RNA, which is biologically active, i.e. which is capable of being translated into a biologically active protein or peptide (or active peptide fragment) or which is active itself (e.g. in posttranscriptional gene silencing or RNAi). The coding sequence is preferably in sense-orientation and encodes a desired, biologically active protein or peptide, or an active peptide fragment. In gene silencing approaches, the DNA sequence is preferably present in the form of an antisense DNA or an inverted repeat DNA, comprising a short sequence of the target gene in antisense or in sense and antisense orientation. “Ectopic expression” refers to expression in a tissue in which the gene is normally not expressed.

A “transcription regulatory sequence” is herein defined as a nucleic acid sequence that is capable of regulating the rate of transcription of a (coding) sequence operably linked to the transcription regulatory sequence. A transcription regulatory sequence as herein defined will thus comprise all of the sequence elements necessary for initiation of transcription (promoter elements), for maintaining and for regulating transcription, including e.g. attenuators or enhancers. Although mostly the upstream (5′) transcription regulatory sequences of a coding sequence are referred to, regulatory sequences found downstream (3′) of a coding sequence are also encompassed by this definition.

As used herein, the term “promoter” refers to a nucleic acid fragment that functions to control the transcription of one or more genes, located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically (e.g. by external application of certain compounds) or developmentally regulated. A “tissue specific” promoter is only active in specific types of tissues or cells, while a “tissue preferred” promoter is preferentially, but not exclusively, active in certain tissues or cells. A “promoter which is active in plants or plant cells” is a promoter which has the capability of initiating transcription in plant cells.

A “meiosis associated promoter” or “meiosis preferred promoter” refers to a promoter that is mainly active during meiosis or during a part of meiosis, preferably during early stages of meiosis (such as early to mid prophase I stages). Preferably, the promoter is not or only very weakly active in somatic cells or in post-meiotic cells.

A “meiosis specific promoter” is active only during meiosis or during parts of meiosis. “Early prophase” refers to leptotene and early zygotene stages of meiotic prophase I, whereas “mid prophase” refers to late zygotene and pachytene stages of meiotic prophase I.

As used herein, the term “operably linked” refers to a linkage of polynucleotide elements in a functional relationship. A nucleic acid is “operably linked” when it is placed into a functional relationship with another nucleic acid sequence. For instance, a promoter, or rather a transcription regulatory sequence, is operably linked to a coding sequence if it affects the transcription of the coding sequence. Operably linked means that the DNA sequences being linked are typically contiguous and, where necessary to join two protein encoding regions, contiguous and in reading frame so as to produce a “chimeric protein”. A “chimeric protein” or “hybrid protein” is a protein composed of various protein “domains” (or motifs) which is not found as such in nature but which a joined to form a functional protein, which displays the functionality of the joined domains (for example DNA binding or repression leading to a dominant negative function). A chimeric protein may also be a fusion protein of two or more proteins occurring in nature. The term “domain” as used herein means any part(s) or domain(s) of the protein with a specific structure or function that can be transferred to another protein for providing a new hybrid protein with at least the functional characteristic of the domain.

The terms “target peptide” refers to amino acid sequences which target a protein to intracellular organelles such as plastids, preferably chloroplasts, mitochondria, or to the extracellular space (secretion signal peptide). A nucleic acid sequence encoding a target peptide may be fused (in frame) to the nucleic acid sequence encoding the amino terminal end (N-terminal end) of the protein.

A “nucleic acid construct” or “vector” is herein understood to mean a man-made nucleic acid molecule resulting from the use of recombinant DNA technology and which is used to deliver exogenous DNA into a host cell. The vector backbone may for example be a binary or superbinary vector (see e.g. U.S. Pat. No. 5,591,616, US2002138879 and WO9506722), a co-integrate vector or a T-DNA vector, as known in the art and as described elsewhere herein, into which a chimeric gene is integrated or, if a suitable transcription regulatory sequence is already present, only a desired nucleic acid sequence (e.g. a coding sequence, an antisense or an inverted repeat sequence) is integrated downstream of the transcription regulatory sequence. Vectors usually comprise further genetic elements to facilitate their use in molecular cloning, such as e.g. selectable markers, multiple cloning sites and the like (see below).

A “host cell” or a “transgenic host cell” or “transformed cell” are terms referring to a new individual cell (or organism) arising as a result of at least one nucleic acid molecule, especially comprising a chimeric gene encoding a desired protein or a nucleic acid sequence which upon transcription yields an antisense RNA or an inverted repeat RNA (or hairpin RNA) for silencing of a target gene/gene family, having been introduced into said cell. The host cell is preferably a plant cell. The host cell may contain the nucleic acid construct as an extra-chromosomally (episomal) replicating molecule, or in one embodiment, comprises the chimeric gene integrated in the nuclear or plastid genome of the host cell.

The term “selectable marker” is a term familiar to one of ordinary skill in the art and is used herein to describe any genetic entity which, when expressed, can be used to select for a cell or cells containing the selectable marker. Selectable marker gene products confer for example antibiotic resistance, or more preferably, herbicide resistance or another selectable trait such as a phenotypic trait (e.g. a change in pigmentation) or a nutritional requirement.

The term “reporter” is mainly used to refer to visible markers, such as green fluorescent protein (GFP), eGFP, luciferase, GUS and the like.

The term “ortholog” of a gene or protein refers herein to the homologous gene or protein found in another species, which has the same function as the gene or protein, but (usually) diverged in sequence from the time point on when the species harbouring the genes diverged (i.e. the genes evolved from a common ancestor by speciation). Orthologs of the tomato mlh1 gene may thus be identified in other plant species based on both sequence comparisons (e.g. based on percentages sequence identity over the entire sequence or over specific domains) and functional analysis.

The terms “homologous” and “heterologous” refer to the relationship between a nucleic acid or amino acid sequence and its host cell or organism, especially in the context of transgenic organisms. A homologous sequence is thus naturally found in the host species (e.g. a tomato plant transformed with a tomato gene), while a heterologous sequence is not naturally found in the host cell (e.g. a tomato plant transformed with a sequence from potato plants). Depending on the context, the term “homolog” or “homologous” may alternatively refer to sequences which are descendent from a common ancestral sequence (e.g. they may be orthologs).

“Stringent hybridisation conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tₘ) for the specific sequences at a defined ionic strength and pH. The Tₘ is the temperature (under defined ionic strength and pH) at which 50% of the target sequence hybridises to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridisations (Northern blots using a probe of e.g. 100 nt) are for example those which include at least one wash in 0.2×SSC at 63° C. for 20 min, or equivalent conditions. Stringent conditions for DNA-DNA hybridisation (Southern blots using a probe of e.g. 100 nt) are for example those which include at least one wash (usually 2) in 0.2×SSC at a temperature of at least 50° C., usually about 55° C., for 20 min, or equivalent conditions. See also Sambrook et al. (1989) and Sambrook and Russell (2001).

“Sequence identity” and “sequence similarity” can be determined by alignment of two peptide or two nucleotide sequences using global or local alignment algorithms. Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length, maximizing the number of matches and minimises the number of gaps. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA. Also, EmbossWin version 2.10.0 can be used, using the program ‘needle’ (which corresponds to GAP) with the same parameters as for GAP above. Alternatively percent similarity or identity may be determined by searching against databases such as FASTA, BLAST, etc.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”. It is further understood that, when referring to “sequences” herein, generally the actual physical molecules with a certain sequence of subunits (e.g. amino acids) are referred to.

“Plant” refers to either the whole plant or to parts of a plant, such as cells, tissue or organs (e.g. pollen, seeds, gametes, roots, leaves, flowers, flower buds, anthers, fruit, etc.) obtainable from the plant, as well as derivatives of any of these and progeny derived from such a plant by selling or crossing. “Plant cell(s)” include protoplasts, gametes, suspension cultures, microspores, pollen grains, etc., either in isolation or within a tissue, organ or organism.

The term “antibody” includes reference to antigen binding forms of antibodies (e.g., Fab, F(ab)2). The term “antibody” frequently refers to a polypeptide substantially encoded by an immunoglobulin gene or immunoglobulin genes, or fragments thereof which specifically bind and recognise an analyte (antigen). However, while various antibody fragments can be defined in terms of the digestion of an intact antibody, one of skill will appreciate that such fragments may be synthesised de novo either chemically or by utilising recombinant DNA methodology. Thus, the term antibody, as used herein, also includes antibody fragments such as single chain Fv, chimeric antibodies (i.e., comprising constant and variable regions from different species), humanised antibodies (i.e., comprising a complementarity determining region (CDR) from a non-human source) and heteroconjugate antibodies (e.g., bispecific antibodies). The term “antigen” includes reference to a substance to which an antibody can be generated and/or to which the antibody is specifically immunoreactive. The specific immunoreactive sites within the antigen are known as epitopes or antigenic determinants. These epitopes can be a linear array of monomers in a polymeric composition—such as amino acids in a protein—or consist of or comprise a more complex secondary or tertiary structure.

“Lycopersicon esculentum” has been officially renamed Solanum lycopersicum, although the unofficial name is still used. These names are synonymous herein.

### DETAILED DESCRIPTION

### Methods of Producing Transgenic Plants and Recombinants According to the Invention

In one embodiment of the invention a method for producing a transgenic plant having an altered frequency of homologous recombination compared to a non-transgenic plant, or another control plant (such as a plant transformed with a control vector) is provided.

It was surprisingly found that overexpression of a nucleic acid sequence encoding an active MLH1 protein under control of a suitable promoter leads to an altered (or modified) meiotic homologous recombination frequency.

Depending on the choice of the promoter, various alterations in recombination frequency can be achieved, as described further below. In a preferred embodiment the frequency of meiotic homologous recombination is significantly increased or significantly decreased by expressing a cDNA or genomic DNA encoding an MLH1 protein under the control of a promoter active in plants. Preferably, a promoter is used which is active in plant cells at least during meiosis or which is meiosis-preferred or meiosis specific (as defined above). Such promoters include for example promoters of genes involved in meiosis, e.g. the ATDMC1 promoter, HvDMC1 promoter or LeDMC1 promoter described in WO98/2843, or the DMC1 promoter obtained from another plant species. Other developmentally regulated or inducible promoters may also be used, as described further below.

A significant alteration (increase or decrease) in the frequency of meiotic homologous recombination can be determined by methods known in the art, such as cytological assays, e.g. Electron Microscopy studies of (average) numbers of late RNs per cell, or by genetic studies using markers. Herein a “significant alteration” (increase or decrease) is preferably an alteration of at least 0.5%, 1%, 2%, 10%, 15%, 20%, 30%, 40%, 50%, 60%, 70%, 80%, 90% or more (e.g. 100%), compared to controls.

In one embodiment the frequency of interfering meiotic homologous recombination is significantly altered, preferably significantly increased or decreased. An increase in interfering meiotic homologous recombination may result in an overall significant increase of meiotic homologous recombination or may result in a concomitant decrease in non-interfering meiotic homologous recombination, without significantly affecting the total frequency of meiotic homologous recombination. Thus, the ratio of interfering to non-interfering meiotic homologous recombination may be altered from that found in the non-transgenic plant species. For example, in non-transgenic cherry tomato, the ratio was found to be about 70:30. The ratio may be modified by overexpression of an MLH1 protein to any other ratio, such as 0:100, 10:90, 20:80, 30:70, 40:60, 50:50, 60:40, 80:20, 90:10, 100:0.

The alteration in the frequency of interfering meiotic homologous recombination may be determined by using a polyclonal or monoclonal anti-MLH1 antibody, which is contacted with meiotic chromosome spreads, or specimen which expose the nuclear chromosomes, in a cytological assay as described in the Examples and below. The number of anti-MLH1 labeled foci (i.e. antibody labeled late RNs) can then be assessed by detecting the label, which is preferably a fluorescent label, detectable by immunofluorescent light microscopy or immuno-Electron Microscopy and quantifiable by image analysis. By determining the difference between interfering and total recombination frequencies, one can calculate the proportion of interfering/non-interfering meiotic homologous recombination and also the proportions with respect to the total. See the Examples. Wild type cherry tomato, for example, was found to have an average of about 1.82 RNs per chromosome and a total of about 21.89 per nucleus (12 chromosomes). Of this total, about 70% of the RNs (about 15.36 RNs/nucleus) represent interfering crossovers, while the remaining 30% of the RNs represent non-interfering crossovers.

In another embodiment, a method for producing a transgenic plant having an altered distribution or positioning of meiotic homologous recombination events on one or more chromosomes. The alteration in positioning may occur in addition to a change in frequency of recombination events, or alternatively without a change in the frequency of meiotic homologous recombination, or of interfering meiotic homologous recombination. A change in distribution may for example result in certain chromosome having a larger number of RNs than normally found on said chromosomes or chromosome sections or arms (e.g. above 2, 3, 4, 5, or more RNs), while other chromosomes may have a lower number (for example no RNs). To detect the location of RNs on chromosomes, preferably a cytological assay using three types of antibodies is used, namely one, that labels late RNs and interfering crossovers (e.g. anti-MLH1 antibodies), one that detects the axial elements of the synaptonemal complexes (e.g. anti-SMC1 or anti-SMC3 antibodies) and one that labels the centromere regions (e.g. anti-CENP-C antibodies). This enables the measurement of chromosome length and identification of the chromosome as well as the location of the centromere and of the RNs on the individual chromosomes.

The method of generating a transgenic plant with the above alterations comprising firstly transforming a plant or plant cell with a nucleotide sequence encoding an MLH1 protein operably linked to a promoter active in plant cells, and secondly regenerating a plant. In one embodiment the nucleotide sequence is preferably not integrated in the plants genome, but remains in the cells on an episomal unit. In another embodiment the chimeric gene is stably integrated into the genome. Both types of transformants can be generated using known methods. For example, if Agrobacterium mediated transformation is used and left and right border sequences are present in the transformation vector at either side of the chimeric gene, integration into the genome will occur. The advantage of not having the MLH1 encoding nucleic acid sequence integrated into the genome is that it can later, after it has altered meiotic homologous recombination in the desired way, be easily removed again by selecting progeny which lacks the episomal unit.

The regenerated transgenic plant may then be used for the production of another plant or a population (or plurality) of plants (or plant seeds) and further, optionally, selecting one or more plants therefrom using various criteria. For example, a plant can be selected having a recombination event between two previously tightly linked loci and where this linkage is now broken. Thus, e.g. rare recombinants may be identified and/or selected for further use.

Thus, the transgenic plant may be used as male or female parent in a cross with another plant of the same species, it may be selfed or cells of the plant may be used to regenerate another plant therefrom. The progeny of a cross or selfing may contain an increased or decreased number of recombinants and/or may have an altered distribution of recombination sites per chromosome or per chromosome set. The method of the invention, therefore, also provides a method for producing a population of recombinant plants (or a population of seeds) having an altered number of recombinants compared to control populations and/or an altered distribution of recombination events. If the frequency of recombination is decreased to zero, the “recombinants” are actually not really recombinants, but are identical to the parental plants.

These plants according to the invention may also be further used or analyzed using molecular methods, in breeding methods, etc. Especially interesting is the identification of rare recombinants, such as recombination in chromosomal cold spots or between two genes of interest that are normally difficult to separate by recombination (linkage drag). If the mlh1 transgene is still present in some of the progeny, it may be crossed-out or removed using for example flp/frt or cre/lox recombination systems, as known in the art. Alternatively, if the transgene was present in an episomal unit, it may be removed by selection of plants/cells lacking the unit.

### Nucleic Acid and Amino Acid Sequences, Chimeric Genes and Vectors

Any nucleic acid sequence encoding an active MLH1 protein or protein variant or fragment may be used for making a chimeric gene, vector and transformed plant or plant cell.

An active MLH1 protein is a protein which shows MLH1 activity in the cell in vivo, i.e. it has biological activity and is therefore able to alter meiotic homologous recombination (frequency and/or distribution) in a transformed plant.

Biological activity (or biological function) can be tested using a variety of known methods, for example by generating a transformed plant overexpressing the gene as described in the Examples and analyzing whether a change in homologous recombination frequency is observed compared to control plants, using for example the cytological assays described herein.

Biological activity may also be determined by assaying the proteins mismatch repair activity. Such methods are known to one of skill in the art and include, but are not limited to, in vitro mismatch repair assays, in vitro mismatch excision assays, nitrocellulose filter binding assays, gel mobility shift assays, helicase assays, and in vivo mutator assays and the like. See WO02/24890.

It is understood that in any transformation experiments a certain degree of variation in the phenotype of transformants is seen, normally due to position effects in the genome and/or due to copy number. A skilled person will know how to compare transformants to one another, e.g. by selecting single copy number events and analysing these. Other methods of determining or confirming in vivo gene/protein function include the generation of knock-out mutants or transient expression studies. Promoter-reporter gene expression studies may also provide information as to the spatio-temporal expression pattern and the role of the protein.

Some MLH1 encoding nucleic acid sequences have already been cloned, such as an Arabidopsis thaliana and a rice mlh1 nucleic acid sequences (WO0224890), which are the only two full length plant sequences available. From a number of other plant species, fragments of MLH1 proteins have been identified, which can be used to isolate full length sequences. Also, SEQ ID NO: 1 (wild type Lemlh1 sequence from tomato) and SEQ ID NO: 2 (expression optimization of SEQ ID NO: 1) and SEQ ID NO: 3 (LeMLH1 amino acid sequence from tomato) are provided herein. Due to the degeneracy of the genetic code, additional nucleic acid sequences encoding the protein of SEQ ID NO: 3 are also provided. These sequences, as well as variants and fragments (see below) encoding functional MLH1 proteins or protein fragments, are used in a preferred embodiment, especially for transformation of plants belonging to the family Solanaceae, especially the genus Solanum (“Solanum” herein includes the genus Lycopersicon), in particular tomato species.

Other putative MLH1 encoding nucleic acid sequences can be identified in silico, e.g. by identifying nucleic acid or protein sequences in existing nucleic acid or protein database (e.g. GENBANK, SWISSPROT, TrEMBL) and using standard sequence analysis software, such as sequence similarity search tools (BLASTN, BLASTP, BLASTX, TBLAST, FASTA, etc.). Especially the screening of plant sequence databases, such as the wheat genome database, etc. for the presence of amino acid sequences or nucleic acid sequences encoding MLH1 proteins is desired. Putative amino acid sequences or nucleic acid sequences can then be selected, cloned or synthesized de novo and tested for in vivo functionality by e.g. overexpression in a host or host cell. Further sequences may be identified using known mlh1 sequences to design (degenerate) primers or probes as described below.

Thus, in principle any MLH1 protein encoding nucleic acid sequence (cDNA, genomic DNA, RNA) may be used. Also included are variants and fragments of mlh1 nucleic acid sequences, such as nucleic acid sequences hybridizing to mlh1 nucleic acid sequences, e.g. to Lemlh1, under stringent hybridization conditions as defined. Variants of mlh1 nucleic acid sequences include nucleic acid sequences which have a sequence identity to SEQ ID NO: 1 (Lemlh1) and/or to SEQ ID NO: 2 (optimized Lemlh1) of at least 50% or more, preferably at least 55%, 60%, 70%, 80%, 90%, 95%, 99%, 99.5%, 99.8% or more, as determined using pairwise alignment using the GAP program using full lengths sequences. Such variants may also be referred to as being “essentially similar” to SEQ ID NO: 1 and/or 2. Fragments include parts of the above mlh1 nucleic acid sequences, which may for example be used as primers or probes or in gene silencing constructs. Parts may be contiguous stretches of at least 15, 20, 21, 100, 200, 450, 500, 1000 or more nucleotides in length. Preferably the mlh1 nucleic acid sequences are of plant origin (i.e. they naturally occur in plant species) or are modified plant sequences.

It is clear that many methods can be used to identify, synthesise or isolate variants or fragments of mlh1 nucleic acid sequences, such as nucleic acid hybridization, PCR technology, in silico analysis and nucleic acid synthesis, and the like. Thus, an MLH1 encoding nucleic acid sequence may be a sequence which is chemically synthesized or which is cloned from any organism (e.g. plant, animal, fungi, yeast), but preferably plant sequences are used, more preferably a sequence originating from a particular plant species is reintroduced into said species (optionally with prior sequence modification, such as codon usage optimization). Thus, in a preferred embodiment, the mlh1 DNA corresponds to, or is a modification/variant of, the endogenous mlh1 DNA of the species which is used as host species in transformation. Thus, a tomato mlh1 cDNA or genomic DNA (or a variant or fragment thereof) is preferably used to transform tomato plants.

Because cloning and vector construction of the wild type Lemlh1 nucleic acid sequence was found to be difficult due to the presence of many different restriction sites, it is a preferred embodiment to modify the wild type nucleic acid sequence so that these undesired restriction sites are removed while the translation of the nucleotide sequence into amino acids remains the same. Thus, for example one, two, three or more recognition sites for different restriction enzymes can be removed/altered to enable cloning in bacterial hosts and/or construction of a vector for transformation. To “remove” these sites the nucleic acid sequence needs to be modified in such a way that the restriction enzymes to be used does not recognize the site, while the translation into amino acids remains the same. This can be done using various methods, such as de novo synthesis of the sequence. As similar problems are likely to occur in the mlh1 nucleic acid sequences of other plant species, it is a general embodiment to provide mlh1 nucleic acid sequence (of any of the above mlh1 nucleic acid sequences and sequence variants) which comprise a reduced number of restriction enzyme recognition sites, preferably wherein at least 2, 3, 4, 5 or more recognition sites of different restriction enzymes have been removed, preferably at least for those restriction enzymes mentioned in the Examples.

Furthermore, for optimized in-planta expression the codon usage of an MLH1 encoding nucleic acid sequence is, in one embodiment, adapted to the preferred codon usage of the host species which is to be transformed. In a preferred embodiment any of the above mlh1 DNA sequences (or variants) are codon-optimized by adapting the codon usage to that most preferred in the host genus or preferably the host species (Bennetzen & Hall, 1982, J. Biol. Chem. 257, 3026-3031; Itakura et al., 1977 Science 198, 1056-1063.) using available codon usage tables (e.g. more adapted towards expression in cotton, soybean corn or rice). Codon usage tables for various plant species are published for example by Ikemura (1993, In “Plant Molecular Biology Labfax”, Croy, ed., Bios Scientific Publishers Ltd.) and Nakamura et al. (2000, Nucl. Acids Res. 28, 292.) and in the major DNA sequence databases (e.g. EMBL at Heidelberg, Germany). Accordingly, synthetic DNA sequences can be constructed so that the same or substantially the same proteins are produced. Several techniques for modifying the codon usage to that preferred by the host cells can be found in patent and scientific literature. The exact method of codon usage modification is not critical for this invention. Other modification, which may optimize expression in plants and/or which make cloning procedures easier may be carried out, such as removal of cryptic splice sites, avoiding long AT or GC rich stretches, etc. (see Examples). Such methods are known in the art and standard molecular biology techniques can be used.

SEQ ID NO: 2 provides an optimized Lemlh1 nucleic acid sequence, which encodes the same amino acid sequence as the wild type Lemlh1 nucleic acid sequence. This sequence was optimized by both removing restriction sites and by codon optimization. A “codon-optimized” sequence preferably has at least about the same GC content or a higher GC content than the genes of the host species into which it is to be introduced. For example, in L. esculentum the GC content of endogenous genes is about 30-40%. The preferred GC contents of MLH1-encoding nucleic acid sequences for transformation of L. esculentum is therefore a GC content of at least 30-40%, preferably above 40%, such as at least 45%, 50%, 55%, 60%, 70% or more. Preferably regions of very high (>80%) or very low (<30%) GC content should be avoided.

Small modifications to a DNA sequence can be routinely made, i.e., by PCR-mediated mutagenesis (Ho et al., 1989, Gene 77, 51-59., White et al., 1989, Trends in Genet. 5, 185-189). More profound modifications to a DNA sequence can be routinely done by de novo DNA synthesis of a desired coding region using available techniques.

Also, the mlh1 nucleic acid sequences can be modified so that the N-terminus of the MLH1 protein has an optimum translation initiation context, by adding or deleting one or more amino acids at the N-terminal end of the protein. Often it is preferred that the proteins of the invention to be expressed in plants cells start with a Met-Asp or Met-Ala dipeptide for optimal translation initiation. An Asp or Ala codon may thus be inserted following the existing Met, or the second codon, Val, can be replaced by a codon for Asp (GAT or GAC) or Ala (GCT, GCC, GCA or GCG). The DNA sequences may also be modified to remove illegitimate splice sites.

In addition to their biological function, “MLH1 proteins” can be defined structurally by the percentage sequence identity over their entire length. MLH1 proteins have a sequence identity of 50% or more over their entire length to SEQ ID NO: 3 (LeMLH1), such as but not limited to at least 40%, 45%, 50%, 55%, 56%, 58%, 60%, 70%, 75%, 80%, 85%, 90%, 95%, 98%, 99%, 99.5,%, 99.8% or more at the amino acid sequence level, as determined using pairwise alignment using the GAP program (with a gap creation penalty of 8 and an extension penalty of 2). Such variants may also be referred to as being “essentially similar” to SEQ ID NO: 3. For example, the Arabidopsis MLH1 protein and the Rice MLH1 protein have 55.9% and 52.9% amino acid sequence identity to LeMLH1, respectively. Preferably proteins having some, preferably 5-10, 20, 30, 50, 100, 200, 300, or more amino acids added, replaced or deleted without significantly changing the protein activity are included in this definition. For example conservative amino acid substitutions within the categories basic (e.g. Arg, His, Lys), acidic (e.g. Asp, Glu), nonpolar (e.g. Ala, Val, Trp, Leu, Ile, Pro, Met, Phe, Trp) or polar (e.g. Gly, Ser, Thr, Tyr, Cys, Asn, Gln) fall within the scope of the invention as long as the activity of the MLH1 protein is not significantly, preferably not, changed. In addition non-conservative amino acid substitutions fall within the scope of the invention as long as the activity of the MLH1 protein is not changed significantly, preferably not changed. Also MLH1 protein fragments and active chimeric MLH1 proteins are encompassed herein. Protein fragments may for example be used to generate antibodies against MLH1 (anti-MLH1 antibodies), as described elsewhere herein. Protein fragments may be fragments of at least about 5, 10, 20, 40, 50, 60, 70, 90, 100, 150, 152, 160, 200, 220, 230, 250, 300, 400, 500, 600, 700 or more contiguous amino acids. Nucleic acid sequences encoding such fragments are also provided, which may for example be used in the construction of gene silencing vectors as described below or for the expression of peptides which can be used to raise antibodies against. Also, the smallest protein fragment which retains activity in vivo in plants is also provided. A nucleic acid sequence encoding such a fragment may be use to generate a transgenic plant as described.

### Chimeric Genes and Vectors According to the Invention

In one embodiment of the invention nucleic acid sequences encoding MLH1 proteins (or variants or fragments) as described above, are used to make chimeric genes, and vectors comprising these for transfer of the chimeric gene into a host cell and production of the MLH1 protein in host cells, such as cells, tissues, organs or whole organisms derived from transformed cell(s).

Host cells are preferably plant cells. Any plant may be a suitable host, such as monocotyledonous plants or dicotyledonous plants, for example maize/corn (Zea species, e.g. Z. mays, Z. diploperennis (chapule), Zea luxurians (Guatemalan teosinte), Zea mays subsp. huehuetenangensis (San Antonio Huista teosinte), Z. mays subsp. mexicana (Mexican teosinte), Z. mays subsp. parviglumis (Balsas teosinte), Z. perennis (perennial teosinte) and Z. ramosa), wheat (Triticum species), barley (e.g. Hordeum vulgare), oat (e.g. Avena sativa), sorghum (Sorghum bicolor), rye (Secale cereale), soybean (Glycine spp, e.g. G. max), cotton (Gossypium species, e.g. G. hirsutum, G. barbadense), Brassica spp. (e.g. B. napus, B. juncea, B. oleracea, B. rapa, etc), sunflower (Helianthus annus), tobacco (Nicotiana species), alfalfa (Medicago sativa), rice (Oryza species, e.g. O. sativa indica cultivar-group or japonica cultivar-group), forage grasses, pearl millet (Pennisetum spp. e.g. P. glaucum), tree species, vegetable species, such as Lycopersicon ssp (recently reclassified as belonging to the genus Solanum), e.g. tomato (L. esculentum, syn. Solanum lycopersicum), potato (Solanum tuberosum) and other Solanum species, such as eggplant (Solanum melongena), tomato (S. lycopersicum, e.g. cherry tomato, var. cerasiforme or current tomato, var. pimpinellifolium), tree tomato (S. betaceum, syn. Cyphomandra betaceae), pepino (S. muricatum), cocona (S. sessiliflorum) and naranjilla (S. quitoense); peppers (Capsicum annuum, Capsicum frutescens), pea (e.g. Pisum sativum), bean (e.g. Phaseolus species), fleshy fruit (grapes, peaches, plums, strawberry, mango) ornamental species (e.g. Rose, Petunia, Chrysanthemum, Lily, Gerbera species), woody trees (e.g. species of Populus, Salix, Quercus, Eucalyptus), fibre species e.g. flax (Linum usitatissimum) and hemp (Cannabis sativa). In one embodiment vegetable species, especially Solanum species (including Lycopersicon species) are preferred.

Thus, for example species of the following genera may be transformed: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Cucumis, Hyoscyamus, Lycopersicon, Nicotiana, Solanum, Malus, Petunia, Digitalis, Majorana, Ciahorium, Helianthus, Lactuca, Bromus, Citrullus, Asparagus, Antirrhinum, Heterocallis, Nemesis, Pelargonium, Panieum, Pennisetum, Ranunculus, Senecio, Salpiglossis, Browaalia, Glycine, Pisum, Phaseolus, cotton, soy and Lolium. A further preference is for each of Cucurbita, Brassica, Lycopersicon, Solanum, Oryza and Zea. A preference is for each of Avena, Medicago, Capsicum, Nicotiana, Lactuca, Pisum, Cucurbita, Brassica, Solanum (including Lycopersicon), Oryza and Zea.

The construction of chimeric genes and vectors for introduction of MLH1 protein encoding nucleic acid sequences into the genome of host cells is generally known in the art. To generate a chimeric gene the nucleic acid sequence encoding a MLH1 protein (or variant or functional fragment) is operably linked to a promoter sequence, suitable for expression in the host cells, using standard molecular biology techniques. The promoter sequence may already be present in a vector so that the mlh1 nucleic sequence is simply inserted into the vector downstream of the promoter sequence. The vector is then used to transform the host cells and the chimeric gene is inserted in the nuclear genome or into the plastid, mitochondrial or chloroplast genome and expressed there using a suitable promoter (e.g., Mc Bride et al., 1995 Bio/Technology 13, 362; U.S. Pat. No. 5,693,507). In one embodiment a chimeric gene comprises a suitable promoter for expression in plant cells, operably linked thereto a nucleic acid sequence encoding a MLH1 protein, protein variant or protein fragment (or fusion protein or chimeric protein) according to the invention, optionally followed by a 3′nontranslated nucleic acid sequence.

The mlh1 nucleic acid sequence, preferably the MLH1 chimeric gene, encoding an functional MLH1 protein, can be stably inserted in a conventional manner into the nuclear genome of a single plant cell, and the so-transformed plant cell can be used in a conventional manner to produce a transformed plant that has an altered phenotype due to the presence of the MLH1 protein in certain cells at a certain time. In this regard, a T-DNA vector, comprising a nucleic acid sequence encoding an MLH1 protein, in Agrobacterium tumefaciens can be used to transform the plant cell, and thereafter, a transformed plant can be regenerated from the transformed plant cell using the procedures described, for example, in EP 0 116 718, EP 0 270 822, PCT publication WO84/02913 and published European Patent application EP 0 242 246 and in Gould et al. (1991, Plant Physiol. 95, 426-434). The construction of a T-DNA vector for Agrobacterium mediated plant transformation is well known in the art. The T-DNA vector may be either a binary vector as described in EP 0 120 561 and EP 0 120 515 or a co-integrate vector which can integrate into the Agrobacterium Ti-plasmid by homologous recombination, as described in EP 0 116 718.

Preferred T-DNA vectors each contain a promoter operably linked to MLH1 encoding nucleic acid sequence between T-DNA border sequences, or at least located to the left of the right border sequence. Border sequences are described in Gielen et al. (1984, EMBO J. 3,835-845). Of course, other types of vectors can be used to transform the plant cell, using procedures such as direct gene transfer (as described, for example in EP 0 223 247, or particle or microprojectile bombardment as described in US2005/055740 and WO2004/092345), pollen mediated transformation (as described, for example in EP 0 270 356 and WO85/01856), protoplast transformation as, for example, described in U.S. Pat. No. 4,684,611, plant RNA virus-mediated transformation (as described, for example in EP 0 067 553 and U.S. Pat. No. 4,407,956), liposome-mediated transformation (as described, for example in U.S. Pat. No. 4,536,475), and other methods such as those described methods for transforming certain lines of corn (e.g., U.S. Pat. No. 6,140,553; Fromm et al., 1990, Bio/Technology 8, 833-839; Gordon-Kamm et al., 1990, The Plant Cell 2, 603-618) and rice (Shimamoto et al., 1989, Nature 338, 274-276; Datta et al. 1990, Bio/Technology 8, 736-740) and the method for transforming monocots generally (PCT publication WO92/09696). For cotton transformation see also WO 00/71733, and for rice transformation see also the methods described in WO92/09696, WO94/00977 and WO95/06722. For sorghum transformation see e.g. Jeoung J M et al. 2002, Hereditas 137: 20-8 or Zhao Z Y et al. 2000, Plant Mol. Biol. 44:789-98). Likewise, selection and regeneration of transformed plants from transformed cells is well known in the art. Obviously, for different species and even for different varieties or cultivars of a single species, protocols are specifically adapted for regenerating transformants at high frequency.

Besides transformation of the nuclear genome, also transformation of the plastid genome, preferably chloroplast genome, is included in the invention. One advantage of plastid genome transformation is that the risk of spread of the transgene(s) can be reduced. Plastid genome transformation can be carried out as known in the art, see e.g. Sidorov V A et al. 1999, Plant J. 19: 209-216 or Lutz K A et al. 2004, Plant J. 37(6):906-13.

The resulting transformed plant can be used in a conventional plant breeding scheme to produce either more transformed plants containing the transgene or to produce recombinant plants/plant populations, preferably lacking the chimeric gene.

The mlh1 nucleic acid sequence is inserted in a plant cell genome so that the inserted coding sequence is downstream (i.e. 3′) of, and under the control of, a promoter which can direct the expression in the plant cell. This is preferably accomplished by inserting the chimeric gene in the plant cell genome, particularly in the nuclear or plastid (e.g. chloroplast) genome.

Preferred promoters include promoters which are active at least during meiosis, more preferably promoters which are meiosis specific or meiosis preferred, as defined. An example of a meiosis preferred promoter is the DMC1 promoter, as this promoter is active during (at least part of) meiosis. Obviously a DMC1 promoter of any species may be used. The DMC1 genes and upstream promoter sequences can be cloned from other species using known methods. Particularly preferred are DMC1 promoters from plant species, such as e.g. tomato, Arabidopsis and barley. Deletion analysis may also be used to identify minimal promoters which are meiosis specific.

Other suitable promoters are the promoters of mlh1 genes themselves or from mlh1 orthologous genes. Equally promoters of genes that are expressed at least during meiosis or during part of meiosis may be identified and used. Suitable other promoters are the MER3 promoter, the MSH4 promoter, SPO11, MSH5, DIF1, etc. Other meiotic plant genes of which the promoter may be suitable are described in T. Schwarzacher, J. Exp. Botany 54 (2003) 11-23.

The spatio-temporal specificity of the promoter and whether it, or a derivative thereof (e.g. using terminal deletion analysis), has a meiosis preferred or meiosis specific expression pattern can be easily tested by operably linking the promoter to a reporter genes using known methods.

Alternatively, the MLH1-encoding nucleic acid sequence may be placed under the control of an inducible promoter that can be induced in meiotic cells. Examples of inducible promoters are the Adh1 promoter which is inducible by hypoxia or cold stress, the Hsp70 promoter which is inducible by heat stress, and the PPDK promoter which is inducible by light. Other examples of inducible promoters are wound-inducible promoters, such as the MPI promoter described by Cordera et al. (1994, The Plant Journal 6, 141), which is induced by wounding (such as caused by insect or physical wounding), or the COMPTII promoter (WO0056897) or the promoter described in U.S. Pat. No. 6,031,151. Alternatively the promoter may be inducible by a chemical, such as dexamethasone as described by Aoyama and Chua (1997, Plant Journal 11: 605-612) and in U.S. Pat. No. 6,063,985 or by tetracycline (TOPFREE or TOP 10 promoter, see Gatz, 1997, Annu Rev Plant Physiol Plant Mol. Biol. 48: 89-108 and Love et al. 2000, Plant J. 21: 579-88). Other inducible promoters are for example inducible by a change in temperature, such as the heat shock promoter described in U.S. Pat. No. 5,447,858, by anaerobic conditions (e.g. the maize ADH1S promoter), by light (U.S. Pat. No. 6,455,760), etc. Obviously, there are a range of other promoters available. Examples of promoters under developmental control include the anther specific promoter 5126 (U.S. Pat. Nos. 5,689,049 and 5,689,051), glob-1 promoter, and gamma-zein promoter.

Constitutive promoters may also be used in certain embodiments. Because these are preferably used in gene silencing approaches, they are described further below.

The mlh1 coding sequence is inserted into the plant genome so that the coding sequence is upstream (i.e. 5′) of suitable 3′ end transcription regulation signals (“3′end”) (i.e. transcript formation and polyadenylation signals). Polyadenylation and transcript formation signals include those of, the nopaline synthase gene (“3′ nos”) (Depicker et al., 1982 J. Molec. Appl. Genetics 1, 561-573.), the octopine synthase gene (“3′ocs”) (Gielen et al., 1984, EMBO J. 3, 835-845) and the T-DNA gene 7 (“3′ gene 7”) (Velten and Schell, 1985, Nucleic Acids Research 13, 6981-6998), which act as 3′-untranslated DNA sequences in transformed plant cells, and others.

A MLH1 encoding nucleic acid sequence can optionally be inserted in the plant genome as a hybrid gene sequence whereby the mlh1 sequence is linked in-frame to a (U.S. Pat. No. 5,254,799; Vaeck et al., 1987, Nature 328, 33-37) gene encoding a selectable or scorable marker, such as for example the neo (or nptII) gene (EP 0 242 236) encoding kanamycin resistance, so that the plant expresses a fusion protein which is easily detectable.

All or part a mlh1 nucleic acid sequence, encoding a MLH1 protein can also be used to transform microorganisms, such as bacteria (e.g. Escherichia coli, Pseudomonas, Agrobacterium, Bacillus, etc.), fungi, viruses, algae or insects. Transformation of bacteria, with all or part of a mlh1 nucleic acid sequence of this invention, incorporated in a suitable cloning vehicle, can be carried out in a conventional manner, preferably using conventional electroporation techniques as described in Maillon et al. (1989, FEMS Microbiol. Letters 60, 205-210.) and WO 90/06999. For expression in prokaryotic host cell, the codon usage of the nucleic acid sequence may be optimized accordingly (as described for plants above). Intron sequences should be removed and other adaptations for optimal expression may be made as known.

For obtaining enhanced expression in monocot plants such as grass species, e.g. corn or rice, an intron, preferably a monocot intron, can be added to the chimeric gene. For example the insertion of the intron of the maize Adh1 gene into the 5′ regulatory region has been shown to enhance expression in maize (Callis et. al., 1987, Genes Develop. 1: 1183-1200). Likewise, the HSP70 intron, as described in U.S. Pat. No. 5,859,347, may be used to enhance expression. The DNA sequence of the mlh1 nucleic acid sequence can be further changed in a translationally neutral manner, to modify possibly inhibiting DNA sequences present in the gene part by means of site-directed intron insertion and/or by introducing changes to the codon usage, e.g., adapting the codon usage to that most preferred by plants, preferably the specific relevant plant genus or species, as described above.

### Gene Silencing

For certain applications, such as stabilization of plant genomes, plant chromosomes or certain allele combinations (e.g. allele pyramiding) or the reconstruction of parental genomes (reverse breeding), it is desired to generate transgenic plants in which the endogenous mlh1 gene or the mlh1 gene family is non functional (T-DNA insertion, mutation), silenced or is silenced in specific cells or tissues of the plant (especially during meiosis). In such plants the frequency of meiotic homologous recombination (especially at least interfering meiotic homologous recombination) is significantly altered, preferably significantly reduced. In this context “significantly reduced” refers to a reduction by at least 1, 2, 3, 5, 10, 20, 30, 50, 70, 90 or preferably 100% compared to control plants (non-transgenic plants or plants transformed with control constructs). Most importantly, the reduction of recombination frequency found in transgenic plants is statistically significant. Thus, the postmeiotic cells (the male and female gametes of such a transgenic plant should maintain the chromosomal makeup of the host plant. This transgenic plant may be used to produce another plant, either by clonal propagation, crossing or selfing, and the like.

The embodiments described above, for methods of making transgenic plants which overexpress an MLH1 protein, essentially also apply to methods for making transgenic plants wherein endogenous mlh1 gene(s) is/are silenced, with the difference that gene silencing vectors are used. “Gene silencing” refers to the down-regulation or complete inhibition of gene expression of one or more target genes. The use of inhibitory RNA to reduce or abolish gene expression is well established in the art and is the subject of several reviews (e.g Baulcombe 1996, Stam et al. 1997, Depicker and Van Montagu, 1997). There are a number of technologies available to achieve gene silencing in plants, such as chimeric genes which produce antisense RNA of all or part of the target gene (see e.g. EP 0140308 B1, EP 0240208 B1 and EP 0223399 B1), or which produce sense RNA (also referred to as co-suppression), see EP 0465572 B1.

The most successful approach so far has however been the production of both sense and antisense RNA of the target gene (“inverted repeats”), which forms double stranded RNA (dsRNA) in the cell and silences the target gene. Methods and vectors for dsRNA production and gene silencing have been described in EP 1068311, EP 983370 A1, EP 1042462 A1, EP 1071762 A1 and EP 1080208 A1.

A vector according to the invention may therefore comprise a transcription regulatory region which is active in plant cells operably linked to a sense and/or antisense DNA fragment of a mlh1 gene according to the invention. Generally short (sense and antisense) stretches of the target gene sequence, such as 17, 18, 19, 20, 21, 22 or 23 nucleotides of coding or non-coding sequence are sufficient. Longer sequences can also be used, such as 100, 200 or 250 nucleotides. Preferably, the short sense and antisense fragments are separated by a spacer sequence, such as an intron, which forms a loop (or hairpin) upon dsRNA formation. Any short stretch of SEQ ID NO: 1 or 2, or of a variant thereof, may be used to make a mlh1 gene silencing vector and a transgenic plant in which one or more mlh1 genes are silenced in all or some tissues or organs or at a certain developmental stage. A convenient way of generating hairpin constructs is to use generic vectors such as pHANNIBAL and pHELLSGATE, vectors based on the Gateway® technology (see Wesley et al. 2004, Methods Mol. Biol. 265:117-30; Wesley et al. 2003, Methods Mol. Biol. 236:273-86 and Helliwell & Waterhouse 2003, Methods 30(4):289-95.), all incorporated herein by reference.

By choosing conserved nucleic acid sequences all mlh1 gene family members in a host plant can be silenced. Encompassed herein are also transgenic plants comprising a promoter active in plants, operably linked to a sense and/or antisense DNA fragment of a mlh1 nucleic acid sequence and exhibiting a mlh1 gene silencing phenotype (a significant alteration in the frequency of meiotic homologous recombination, preferably in the frequency of interfering meiotic homologous recombination).

The promoter may be either a meiosis preferred or meiosis specific or an inducible promoter, as described above, or a constitutive promoter. Suitable constitutive promoters include: CabbB-S (Franck et al., 1980, Cell 21, 285-294) and CabbB-JI (Hull and Howell, 1987, Virology 86, 482-493), promoters from the ubiquitin family (e.g. the maize ubiquitin promoter of Christensen et al., 1992, Plant Mol. Biol. 18, 675-689, EP 0 342 926, see also Cornejo et al. 1993, Plant Mol. Biol. 23, 567-581), the gos2 promoter (de Pater et al., 1992 Plant J. 2, 834-844), the emu promoter (Last et al., 1990, Theor. Appl. Genet. 81, 581-588), Arabidopsis actin promoters such as the promoter described by An et al. (1996, Plant J. 10, 107.), rice actin promoters such as the promoter described by Zhang et al. (1991, The Plant Cell 3, 1155-1165) and the promoter described in U.S. Pat. No. 5,641,876 or the rice actin 2 promoter as described in WO070067; promoters of the Cassava vein mosaic virus (WO 97/48819, Verdaguer et al. 1998, Plant Mol. Biol. 37, 1055-1067), the pPLEX series of promoters from Subterranean Clover Stunt Virus (WO 96/06932, particularly the S7 promoter), a alcohol dehydrogenase promoter, e.g., pAdh1S (GenBank accession numbers X04049, X00581), and the TR1′ promoter and the TR2′ promoter (the “TR1′promoter” and “TR2′promoter”, respectively) which drive the expression of the 1′ and 2′ genes, respectively, of the T-DNA (Velten et al., 1984, EMBO J. 3, 2723-2730), the Figwort Mosaic Virus promoter described in U.S. Pat. No. 6,051,753 and in EP426641, histone gene promoters, such as the Ph4a748 promoter from Arabidopsis (PMB 8: 179-191), Smas promoter, the cinnamyl alcohol dehydrogenase promoter (U.S. Pat. No. 5,683,439), the Nos promoter, the pEmu promoter, the rubisco promoter, the GRP1-8 promoter or others.

Also in this application, the chimeric gene may be introduced stably into the host genome or may be present as an episomal unit.

### Plants and Plant Seeds According to the Invention

Transgenic plants, plant cells, tissues or organs are provided, obtainable by the above methods. These plants are characterized by the presence of a chimeric gene in their cells or genome and/or by having an altered recombination frequency, and/or by having an altered positioning/distribution of recombination events. Any change in recombination frequency is measurable using e.g. cytological assays (e.g. as described herein or as described by Sherman and Stack, 1995, supra), genetic marker analysis, selection and reporter genes, phenotypic markers, and the like.

Transformants expressing high, moderate or low levels of the MLH1 protein (or of the sense and/or antisense transcript in silenced plants) can be selected by e.g. analysing copy number (Southern blot analysis), mRNA transcript levels (e.g. Northern blot analysis or RT-PCR using mlh1 primer pairs or flanking primers) or by analysing the presence and level of MLH1 protein, e.g. in developing flower organs during meiosis (e.g. SDS-PAGE followed by Western blot analysis; ELISA assays, immunocytological assays, etc). The expression level of the mlh1 chimeric gene will depend not only on the strength and specificity of the promoter, but also on the position of the chimeric gene in the genome.

It is thought that the expression level may influence the frequency of homologous recombination and the ratio of interfering and non-interfering crossovers. A skilled person can, however, easily identify plants having the desired change in recombination frequency and/or positioning, optionally without having undesired effects. Thus, by testing various promoters and analyzing a variety of recombinant plants transformed with the same construct (i.e. “transformation events”), the desired plants can be identified and selected for further use. The same applies for plants transformed with a gene silencing construct, where a suitable construct and transformation event can easily be selected using routine methods.

Also a population of plants and a population of plant seeds is provided which is obtainable by using the recombinant plant as a male and/or female parent. The population is characterized by having either an increased frequency/percentage of recombinants or by having a reduced frequency/percentage of recombinants, and/or an altered distribution of recombination events. Individual plants may be selected and used for further breeding methods or seed production methods.

Preferably the plant population sizes required to find a desired recombinant are significantly reduced.

Breeding procedures are known in the art and are described in standard text books of plant breeding, i.e., Allard, R. W., Principles of Plant Breeding (1960) New York, N.Y., Wiley, pp 485; Simmonds, N. W., Principles of Crop Improvement (1979), London, UK, Longman, pp 408; Sneep, J. et al., (1979) Tomato Breeding (p. 135-171) in: Breeding of Vegetable Crops, Mark J. Basset, (1986, editor), The Tomato crop: a scientific basis for improvement, by Atherton, J. G. & J. Rudich (editors), Plant Breeding Perspectives (1986); Fehr, Principles of Cultivar Development-Theory and Technique (1987) New York, N.Y., MacMillan.

### Isolated Nucleic Acids and Proteins According to the Invention

In one embodiment novel mlh1 nucleic acid and MLH1 amino acid sequences are provided, as well as vectors comprising these and methods of using these. The isolated sequences and vectors have already been described in the methods above, but are also an embodiment as such. In particular, SEQ ID NO's 3 (LeMLH1), fragments and variants thereof are provided (as well as chimeric genes and vectors comprising these) and nucleic acid sequences encoding these, such as SEQ ID NO: 1 and 2, and fragments and variants thereof (as well as chimeric genes and vectors comprising these), are provided. In a preferred embodiment the codon optimized sequences of wild type mlh1 nucleic acid sequence are provided (see above), which are particularly suitable for overexpression in plants.

Also provided are sequences suitable for raising antibodies for use in cytological assays, as described herein below.

Antibodies According to the Invention and their Use

The MLH1 proteins according to the invention (including fragments and variants, as defined above) may be used to raise mono- or polyclonal antibodies, which may for example be used for the detection of MLH1 proteins in plant samples (immunochemical analysis methods and kits). Such antibodies are especially suitable for (a) determining the number of late recombination nodules in plant cell nuclei that represent interfering crossovers, and thereby the frequency of interfering meiotic homologous recombination per chromosome or per cell and/or (b) the location or distribution of late RNs representing interfering crossovers among cells, and among and along chromosomes.

For purpose (a) an antibody is required which specifically labels the late RNs representing interfering crossovers, such as an anti-MLH1 antibody, which is able to label MLH1 in chromosome spreads. It was found that anti-MLH1 antibodies are specific for only a fraction of the total late RNs, namely the RNs representing interfering crossovers. Other antibodies which specifically label the late RNs representing interfering crossovers may be identified. Without limiting the scope of the invention, it is presumed that anti-MLH3 antibodies, anti-Mer3 antibodies, anti-Msh4 antibodies and anti-Msh5 antibodies also are specific for RNs representing interfering crossovers.

For purpose (b) at least three antibodies are required, one that labels late RNs representing interfering crossovers (e.g. anti-MLH1 antibody), one that labels the axial elements of the synaptonemal complexes (e.g. anti-SMC1 and/or anti-SMC3 antibodies) and one that labels the centromeric regions (e.g. anti-CENP-C).

Standard methods for raising polyclonal or monoclonal antibodies are used, as described by Harlow and Lane (1988, ISBN 0-87969-314-2, Antibodies—A laboratory manual). For example, a peptide comprising a desired epitope is expressed in a host cell or synthesised, purified and injected into an animal (mouse, rabbit, rat, etc.), which is bled and from the blood the antibodies are recovered.

In one embodiment an anti-MLH1 antibody is provided, which is raised against at least 5, 10, 20, 50, 100, 150, 160, 200, or more consecutive amino acids of SEQ ID NO: 3 or of a variant of SEQ ID NO: 3, as defined above. In one embodiment the anti-MLH1 antibody is raised against SEQ ID NO: 4, or against a fragment of SEQ ID NO: 4, whereby the fragment comprising at least 5, 10, 20, 50, 100, 150 or more consecutive amino acids of amino acids 37-195 of SEQ ID NO: 4 (which correspond to amino acids 443-601 of SEQ ID NO: 3).

Preferably, an antibody is raised against a C-terminal fragment of SEQ ID NO: 3 or a variant of SEQ ID NO: 3, as defined above. The C-terminal was found to be particularly suitable for eliciting the production of strong and specific anti-MLH1 antibodies. The “C-terminal MLH1 region” refers herein to about amino acid 400 to the end of an MLH1 protein or variant thereof. The length of the C-terminal depends on the total length of the protein. For LeMLH1 the C-terminal region is thus 201 amino acids, while for Arabidopsis and rice MLH1 proteins it is longer, as these proteins are longer. A fragment thereof comprises at least 5, 10 20, 50, 100, 150, 200 or more consecutive amino acids of the C-terminal region.

Alternatively, other parts of the MLH1 protein or variant may be used, such as the N-terminal region. For example an antibody raised against amino acids 1-193 of SEQ ID NO: 3 also worked.

The use of anti-MLH1 antibodies, and of other antibodies which specifically label interfering crossovers, for the detection and/or quantification of late RNs in plant cells is one embodiment of the invention. Also, the use in the cytological assays described below is encompassed herein.

Further provided are antibodies, which are suitable for detecting the axial elements of SCs and centromeres in plant cell nuclei, respectively, especially in the cytological assays and kits described herein. Such antibodies include anti-SMC1, anti-SMC3 and anti-CENP-C antibodies. These are raised against SMC1, SMC3 and CENP-C protein fragments of plant proteins. For example, amino acid and nucleic acid fragments of LeSMC1 (amino acids 46-293 of SEQ ID NO: 6 and nucleotides 136-817 of SEQ ID NO: 7) and LeSMC3 (amino acids 37-318 of SEQ ID NO: 10 and nucleotides 108-954 of SEQ ID NO: 11) and LeCENP-C (amino acids 37-209 of SEQ ID NO: 8 and nucleotides of SEQ ID NO: 109-630) are provided herein, which may be used to raise antibodies.

Thus, in one embodiment an anti-SMC1 antibody is provided, which is raised against at least 5, 10, 20, 50, 100, 150, 200 or more consecutive amino acids of amino acids 46-293 of SEQ ID NO: 6 (259 N-terminal amino acid sequence of LeSMC1), or of an amino acid sequence having at least 50, 60, 70, 80, 90, 95, 98 or more amino acid identity to amino acids 46-293 of SEQ ID NO: 6. In one embodiment the anti-SMC1 antibody is raised against SEQ ID NO: 6, or against a fragment of SEQ ID NO: 6, whereby the fragment comprising at least 5, 10, 20, 50, 100, 200 or more consecutive amino acids of amino acids 46-293 of SEQ ID NO: 6. Alternatively, it may be raised against any fragment of a plant SMC1 protein. Full length SMC1 proteins may be cloned and sequenced from any plant species and the sequence may be used to raise antibodies.

In one embodiment an anti-SMC3 antibody is provided, which is raised against at least 5, 10, 20, 50, 100, 150, 200 or more consecutive amino acids of amino acids 37-318 of SEQ ID NO: 10, or of an amino acid sequence having at least 50, 60, 70, 80, 90, 95, 98 or more amino acid identity to amino acids 37-318 of SEQ ID NO: 10. In one embodiment the anti-SMC3 antibody is raised against SEQ ID NO: 10, or against a fragment of SEQ ID NO: 10, whereby the fragment comprising at least 5, 10, 20, 50, 100, 200 or more consecutive amino acids of amino acids 37-318 of SEQ ID NO: 10. Alternatively, it may be raised against any fragment of a plant SMC3 protein. Full length SMC3 proteins may be cloned and sequenced from any plant species and the sequence may be used to raise antibodies.

In yet another embodiment an anti-CENP-C antibody is provided, which is raised against at least 5, 10, 20, 50, 100, 150, or more consecutive amino acids of amino acids 37-209 of SEQ ID NO: 8 (amino acid sequence of the C-terminus of LeCENP-C), or of an amino acid sequence having at least 60, 70, 80, 90, 95, 98 or more amino acid identity to amino acids 37-209 of SEQ ID NO: 8. In one embodiment the anti-CENP-C antibody is raised against SEQ ID NO: 8, or against a fragment of SEQ ID NO: 8, whereby the fragment comprising at least 5, 10, 20, 50, 100, 150 or more consecutive amino acids of SEQ ID NO: 8. Alternatively, it may be raised against any fragment of a plant CENP-C protein. Full length CENP-C proteins may be cloned and sequenced from any plant species and the sequence may be used to raise antibodies.

The antibodies provided are especially useful in the cytological assays described herein. It is understood that the nucleic acid and amino acid sequences, and variants thereof, suitable for raising the above antibodies are also an embodiment of the invention.

### Cytological Assays According to the Invention and Use of Anti-MLH1 Antibodies

Two types of cytological assays are provided and the use of at least one antibody that label late RNs representing interfering crossovers, e.g. anti-MLH1 antibodies, in these assays.

It is an embodiment to use an antibody that label late RNs representing interfering crossovers, e.g. anti-MLH1 antibody, in a cytological assay for the determination of the frequency of interfering meiotic homologous recombination events in plant cells, said method comprising:

(a) preparing a specimen of meiotic pachytene cells/nuclei of a plant,
(b) contacting said specimen with at least an antibody that label late RNs representing interfering crossovers, preferably anti-MLH1 antibody, and optionally with an antibody that labels the axial elements of synaptonemal complexes, e.g. anti-SMC1 or anti SMC3 antibody, and/or an antibody that labels the centromeric region, e.g. anti-CENP-C antibody, and optionally counterstaining chromosomal DNA with DAPI, and
(c) determining the number of MLH1-foci per cell, preferably using light microscopy or electron microscopy.

It is another embodiment to use antibodies that label late RNs representing interfering crossovers, e.g. of an anti-MLH1 antibody, in a cytological assay for the determination of the location and distribution of interfering meiotic homologous recombination events in plant cells, said method comprising:

(a) preparing a specimen of meiotic pachytene cells/nuclei of a plant,
(b) contacting said specimen simultaneously or consecutively with at least an antibody that label late RNs representing interfering crossovers, e.g. an anti-MLH1 antibody, with an antibody that labels the axial elements of synaptonemal complexes, e.g. anti-SMC1 or anti SMC3 antibody, and with an antibody that labels the centromeric region, e.g. anti-CENP-C antibody, and optionally counterstaining chromosomal DNA with DAPI, and
(c) determining the number of labeled MLH1-foci per cell, preferably using light microscopy or electron microscopy.

Step (a) in both assays uses standard methods for preparing specimen of plant cell nuclei, e.g. chromosome spreading techniques as described in the Examples and by Sherman and Stack (1995). Anthers are harvested and at least one anther is used to check the meiotic stage, by squashing and e.g. aceto-orcein staining. The stage is preferably mid prophase I, most preferably pachytene. If the stage is suitable, other anthers are used to isolate pollen mother cells, prepare protoplasts and chromosome spreads. Chromosome spreads are then contacted with one or more antibodies, either consecutively or in combination/simultaneously. Specimen, which allow access of the antibodies to the chromosomes, can also be prepared using other methods known in the art.

The immunocytological labeling method (immunofluorescence) preferably makes use of fluorescent compounds (fluorochromes), which can be detected using light microscopy/fluorescent microscopy. The fluorescent compound may be either covalently attached to the “test antibody” (e.g. anti-MLH1) directly (direct test) or, preferably to a second antibody, whereby the second antibody is specific for the first test antibody (indirect test). The second antibody may, thus, be labeled/conjugated with a fluorescent compound and may bind the test antibody.

Suitable fluorescent compounds are widely known, e.g. FITC (fluorescien isothiocyanate), TR (Texas Red), AMCA.

The fluorescence is scored by image analysis and quantified, using methods known in the art. The images may be superimposed, so that the centromeres, SC axial elements and MLH1 foci become visible on one image.

If the relative proportions of RNs representing interfering crossovers to RNs representing non-interfering crossovers are to be determined, it is preferred that in addition the ultrastructural detection of RNs (Sherman and Stack, 1995) and/or genetic marker analysis is carried out, to determine the total recombination frequency.

These cytological assays are suitable for analyzing transformed, non-transformed or recombinant plants, as well as the influence of various factors on meiotic homologous recombination frequencies and distribution of RNs on chromosomes. The effect of overexpressing or silencing one or more genes, the effect of mutations, and the effect of chromosomal abnormalities and imperfect homology may be analyzed in the same way.

### Non-Transgenic Methods and Plants

Alternatively, non-transgenic plants or plant cells comprising either non-functional alleles of mlh1 or increased expression of endogenous mlh1 genes may be identified. It is also an embodiment of the invention to use non-transgenic methods, e.g. mutagenesis systems such as TILLING (Targeting Induced Local Lesions IN Genomics; McCallum et al., 2000, Nat Biotech 18:455, and McCallum et al. 2000, Plant Physiol. 123, 439-442, both incorporated herein by reference) and selection to generate plant lines which produce lower levels or higher levels of one or more MLH1 proteins according to the invention. Without limiting the scope of the invention, it is believed that such plants could comprise point/deletion mutations in the gene or in the promoter. Mutations in the promoter in regions that are binding sites for repressor proteins would make the host MLH1 gene constitutive or higher in expression. TILLING uses traditional chemical mutagenesis (e.g. EMS mutagenesis) followed by high-throughput screening for mutations (e.g. using Cel 1 cleavage of mutant-wildtype DNA heteroduplexes and detection using a sequencing gel system), see e.g. Henikoff et al. Plant Physiology Preview May 21, 2004. Thus, non-transgenic plants, seeds and tissues comprising an enhanced mlh1 gene expression in one or more tissues and methods for generating and identifying such plants is encompassed herein.

The method comprises in one embodiment the steps of mutagenizing plant seeds (e.g. EMS mutagenesis), pooling of plant individuals or DNA, PCR amplification of a region of interest, heteroduplex formation and high-throughput detection, identification of the mutant plant, sequencing of the mutant PCR product. It is understood that other mutagenesis and selection methods may equally be used to generate such mutant plants. Seeds may for example be radiated or chemically treated and the plants screened for a modified recombination frequency.

In another embodiment of the invention, the plant materials are natural populations of the species or related species that comprise polymorphisms or variations in DNA sequence at the MLH1 orthologous coding and/or regulatory sequence. Mutations in the MLH1 gene target can be screened for using a ECOTILLING approach (Henikoff et al 2004, supra). In this method natural polymorphisms in breeding lines or related species are screened for by the above described TILLING methodology, in which individual or pools of plants are used for PCR amplification of the MLH1 target, heteroduplex formation and high-throughput analysis. This can be followed up by selecting of individual plants having the required mutation that can be used subsequently in a breeding program to incorporate the desired MLH1-orthologous allele to develop the cultivar with desired trait.

In a further embodiment non-transgenic mutant plants which produce lower levels of MLH1 protein in one or more tissues are provided, or which completely lack MLH1 protein in specific tissues or which produce a non-functional MLH1 protein in certain tissues, e.g. due to mutations in one or more endogenous MLH1 alleles. For this purpose also methods such as TILLING may be used. Seeds may be mutagenized using e.g. radiation or chemical mutagenesis and mutants may be identified by detection of DNA polymorphisms using for example CEL 1 cleavage. Especially, mutants which comprise mutations in one or more mlh1 alleles are provided. Non-functional mlh1 alleles may be isolated and sequenced or may be transferred to other plants by breeding methods.

Mutant plants can be distinguished from non-mutants by molecular methods, such as the mutation(s) present in the DNA, MLH1 protein levels, mlh1 RNA levels etc, and by the modified phenotypic characteristics.

The non-transgenic mutants may be homozygous or heterozygous for the mutation conferring the enhanced expression of the endogenous mlh1 gene(s) or for the mutant mlh1 allele(s).

### Sequences

SEQ ID NO 1: mlh1 cDNA from tomato (wild type)
SEQ ID NO 2: mlh1 cDNA (codon optimized tomato sequence)
SEQ ID NO 3: MLH1 amino acid sequence of tomato
SEQ ID NO 4: amino acid sequence used to raise anti-LeMLH1 antibodies
SEQ ID NO 5: nucleic acid sequence used to raise anti-LeMLH1 antibodies
SEQ ID NO 6: amino acid sequence used to raise anti-LeSMC1 antibodies
SEQ ID NO 7: nucleic acid sequence used to raise anti-LeSMC1 antibodies
SEQ ID NO 8: amino acid sequence used to raise anti-LeCENP-C antibodies
SEQ ID NO 9: nucleic acid sequence used to raise anti-LeCENP-C antibodies
SEQ ID NO 10: amino acid sequence used to raise anti-LeSMC3 antibodies
SEQ ID NO 11: nucleic acid sequence used to raise anti-LeSMC3 antibodies
SEQ ID NO 12: Sequence comprising AtDMC1 promoter
SEQ ID NO 13: Arabidopsis MLH1 amino acid sequence (AtMLH1)
SEQ ID NO 14: Rice MLH1 amino acid sequence (OsMLH1)
SEQ ID NO 15-33 Amino acid fragments of other (putative) MLH1 proteins

### FIGURE LEGENDS

FIG. 1: Frequency distribution plot of the number of MLH1 foci per nucleus as observed by immunofluorescence (IF) (peaking curve), compared to the respective predicted Poisson distribution (flat curve).

FIG. 2: The inter-RN distances are plotted against the relative frequencies of those distances; the symbols represent the observations in Table 10 from Sherman and Stack (1995), and the lines show the best fits to the gamma distribution. If the inter-RN distances can be fitted to a gamma distribution, this means that there is interference between RNs. n is a parameter in the formula for the gamma distribution. The table to the right shows for which n-values we obtained the best fits. If the RNs were distributed randomly along the chromosomes, n would equal 1; if n>1, then there is interference between RNs, and the higher n is, the stronger interference is. For further calculations we assumed that late RNs along tomato chromosomes display a low level of interference, with n=3.

FIG. 3: Frequency distribution of distances between late RNs (interval sizes) on the long arm of Chromosome #1. The bars represent data from Sherman and Stack, 1995, Table 10; the best fit to the gamma distribution was obtained for n=2.9). Interval sizes are given in arbitrary units.

FIG. 4: Expected frequency distribution of inter-RN distances. The horizontal axis is drawn to scale with that of FIG. 3. The bars represent the expected distribution of distances between MLH1 foci only, for 1.4 foci/long arm and n=7. This distribution is similar to the inventor's observations, and differs clearly from that in FIG. 3: the peak is further to the right, and there are no very small interfocus distances.

In simulations the inventors found that if the interference-insensitive RNs are placed entirely independently of the MLH1 foci, there will always be more small intervals than observed. An example is shown by the brown/dark line, which represents the expected distribution of distances between RNs (MLH1 positive and negative), if on average 0.63 non-interfering, MLH1-negative RNs were placed per long arm of chromosome 1 in addition to on average 1.4 MLH1 foci per long arm, in such a way that the MLH1-positive RNs do not influence the position of MLH1 negative RNs and vice versa. This would yield far more small interfocus distances than Sherman and Stack found (Sherman and Stack, 1995).

However, if one assumes that the MLH1 positive, high-interference RNs and the MLH1-negative RNs are not placed entirely independently, but originate from the same population of precursors, and that these precursors display already a low level of interference (n=2), we get a similar distribution of inter-RN distances as obtained by Sherman and Stack (yellow/light line).

FIG. 5: Relative frequency distribution of the number of MLH1 foci per nucleus of control (hashed) and MLH1 overexpressing plant no. 10 (filled). Vertical black bars indicate the average number of MLH1 foci for both populations. The numeric values next to the average bars represent the average values. The difference between the two populations is expressed as a percentage of the control population above the double arrow.

FIG. 6: Relative frequency distribution of the number chiasmata per nucleus of control (hashed) and MLH1 overexpressing plant no. 10 (filled). Vertical black bars indicate the average number of chiasmata for both populations. The numeric values next to the average bars represent the average values. The difference between the two populations is expressed as a percentage of the control population above the double arrow.

Unless stated otherwise in the Examples, all recombinant DNA techniques are carried out according to standard protocols as described in Sambrook et al. (1989) Molecular Cloning: A Laboratory Manual, Second Edition, Cold Spring Harbor Laboratory Press, and Sambrook and Russell (2001) Molecular Cloning: A Laboratory Manual, Third Edition, Cold Spring Harbor Laboratory Press, NY; and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Croy, jointly published by BIOS Scientific Publications Ltd (UK) and Blackwell Scientific Publications, UK.

### EXAMPLES

### 1. Tomato mlh1 Gene and Proteins

Tomato cDNA clones encoding MLH1 were isolated and sequenced. The cDNA and amino acid sequences are shown in SEQ ID NO: 1 and SEQ ID NO: 3.

### 2. Antibody Production

The isolated tomato cDNA clones encoding MLH1 were used for anti-MLH1 antibody production. C-terminal, middle and N-terminal amino acid stretches were used to raise antibodies in rabbit. Antibodies raised against the MLH1 C-terminal gave the best signal, although antibodies raised against the N-terminal part also gave a signal, but weaker. The middle part of the protein seemed less suitable for raising antibodies. The antibodies used in the further assays were antibodies raised against SEQ ID NO: 4 (encoded by SEQ ID NO: 5), which comprises C-terminal tomato MLH1 amino acids from the amino acid at position 443 to amino acid 601.

In addition tomato cohesin SMC1 (LeSMC1), SMC3 (LeSMC3) and tomato kinetochore protein CENP-C (LeCENP-C) amino acid sequence stretches were used to prepare antibodies recognizing these proteins.

SEQ ID NO: 6 (encoded by SEQ ID NO: 7) was used to raise anti-LeSMC1 antibodies in rabbit. SEQ ID NO: 6 comprises the N-terminal amino acids of LeSMC1 from amino acid 46 to 293.

SEQ ID NO: 8 (encoded by SEQ ID NO: 9) was used to raise anti-LeCENP-C antibodies in rabbit. SEQ ID NO: 8 comprises 173 amino acids of the C-terminus of LeCENP-C

SEQ ID NO: 10 (encoded by SEQ ID NO: 11) was used to raise anti-LeSMC3 antibodies in rabbit. SEQ ID NO: 10 comprises the N-terminal amino acids of LeSMC3 from amino acid 37-318.

Antibodies were raised in rabbit using standard methods, see Ed Harlow & David Lane, Antibodies—A laboratory manual (1988) Cold Spring Harbor Laboratory—ISBN 0-87969-314-2.

The anti-SMC1 and anti-SMC3 antibodies labeled the axial elements of SCs, whereas anti-MLH1 labeled intense foci along pachytene SCs. The anti-CENP-C antibody strongly labeled the centromeric region on spread preparations of pachytene tomato meiocytes.

This is the first time anti-MLH1 antibodies have been used in plants, to label plant MLH1-proteins and to determine frequency and position/location of RNs on individual chromosomes (see below).

### 3. Cytological Assay

### Cherry Tomato Spreads Preparation Procedure

The chromosome spreading procedure was adapted from Sherman and Stack, 1995 (supra).

### Digestion Medium

1 mL of 2.8 mM KH2PO4 (Merk, MW 136.09, 38.1 mg/100 mL)
1 mL of 0.5 mM acid PIPES (Merk, MW 302.36, 15.12 mg/100 mL)
1 mL of a 1% potassium dextran sulfate prepared freshly from powder (Calbiochem, MW 1500, 10 mg/mL)
1 mL of 2.5 mM CaCl2 (Merk, MW 147.02, 36.75 mg/100 mL CaCl2.2H2O)
1 mL MilliQ water

Mix all solution together and add 0.7 M mannitol (637.7 mg/5 mL) and 1% PVP (44,000 in MW, 50 mg/5 mL). Dissolve completely and adjust pH to 5.1 with 0.1 N KOH.

To 1 mL of digestion medium an enzyme cocktail was added and the solution was gently mixed to fully dissolve the enzymes

### Bursting Medium

### 0.05% Nonidet P-40

### 0.1% BSA

0.001% Potassium dextran sulfate

### The Following Spreading Procedure was Used

Dissect the flower bud under the dissecting microscope.

Collect one anther, measure the length (2.1 mm for pachytene) and cut the tip on a clean glass slide.

Squeeze the PMCs out of the anther and apply immediately one droplet of a 2% aceto-orcein solution. Cover with a coverslip.

Flame for a few seconds using an alcohol lamp, this helps getting the cells loose.

Apply a paper tissue over the coverslip and tap the coverslip with the blunt end of a dissecting needle.

Flame the slide on an alcohol lamp and observe with phase contrast.

If the staging is OK, collect the 4 remaining anthers and transfer them to a depression slide containing 200 μL of digestion medium.

Cut the anther tips and squeeze gently the Pollen Mother Cells (PMCs) out in the digestion medium (if PMCs disperse in the digestion medium, they are probably in later stages than pachytene, extend the digestion time until the PMCs are completely translucent).

Incubate in the digestion medium for >10 min. (15 min. recommended) at room temperature in a wet chamber.

Using a siliconized micro-pipette (obtained by pulling a borosilicate capillary tube in the flame), collect up to 4 rods of cells in a volume of digestion medium as little as possible (at most 0.5 μL).

Expel the cells in a 10 μL droplet of bursting medium hanging at the tip of a micro-pipette.

Immediately apply the droplet of bursting medium containing the cells on a 10 μL droplet of 4% PFA in PBS pH 7.4 (Final PFA concentration on slide 2%) placed in the centre of a glow-discharged slide.

Allow the cells to swell and burst and allow the slides to completely air-dry

Check if the spreads are OK with phase contrast optics.

Wash the slides 5 min. in 0.4% Photoflo 200 in water.

Wash the slides 2 times 5 min. in MilliQ water.

Allow the slides to air-dry in a tube rack.

Slides are wrapped in aluminum foil and stored at −72° C. until use.

### Immunolabeling Procedure for MLH1 Foci and SMC

Slides are blocked for 30 min. at room temperature with 600 μL of a 3% BSA, 0.1% Triton X-100 in PBS sterile-filtered solution supplemented with 0.01% sodium azide, pH 7.4

Slides are incubated 1 hr at 37° C., 48 hours at 4° C. and 1 hr at 37° C. in a wet chamber with 100 μL of the RabαLeMLH1 C-term diluted 1:50 in blocking.

Slides are covered with a coverslip during the incubation.

Slides are washed 3 times 5 min. in filtered PBS at room temperature.

Slides are incubated for >2 hours at 37° C. in a wet chamber with 100 μL/slide of GαRab-FITC-Fab diluted 1:200 in blocking buffer.

Slides are covered with a coverslip during the incubation.

Slides are washed 3 times 5 min. in filtered PBS at room temperature.

Slides are incubated 1 hr at 37°, o/n at 4° C. and 1 hr at 37° C. in a wet chamber with 100 μL/slide of Rabbit anti-LeSMC1 N-term diluted 1:50 in blocking buffer.

Slides are covered with a coverslip during the incubation.

Slides are washed 3 times 5 min in filtered PBS at room temperature.

Slides are incubated 2 hours at 37° C. in a wet chamber with 100 μL/slide of GαRab-TR diluted 1:100 in blocking buffer.

Slides are covered with a coverslip during the incubation.

Slides are washed 3 times 5 min with filtered PBS at room temperature.

Slides are briefly rinsed in FITC buffer.

Slides are mounted in Vectashield containing 1 μg/mL DAPI and sealed with clear fingernail polish.

### Immunolabeling Procedure for CENP-C

Slides are soaked in PBS for ˜15 min. to soften the fingernail polish.

The fingernail polish is peeled off with thin tweezers.

The slides are washed further until the coverslip comes off.

Slides are washed extensively in PBS (>3 times 5 min.).

Slides are blocked for 30 min. with 600 μL/slide blocking buffer.

Slides are incubated with 100 μL/slide of RabαLeCENP-C diluted 1:100 in blocking for 1 hr at 37° C., o/n at 4° C. and 1 hr at 37° C. in a wet chamber.

Slides are washed 3 times 5 min. in filtered PBS.

Slides are incubated for >2 hrs with 100 μL/slide of GαRab-FITC-Fab diluted 1:200 in blocking buffer.

Slides are washed 3 times 5 min. in blocking buffer.

Slides are quickly washed in FITC buffer.

Slides are mounted in Vectashield containing 1 μg/mL DAPI and sealed with clear fingernail polish.

### Post-Processing

After observation, image enhancement, manipulation and registration is carried out with Adobe Photoshop 7 and Image J. Measurements are carried out with a home-written macro for the image analysis program Object Image. Data analysis is carried out with Microsoft Excel 2003, GraphPad Prism 4, GenStat 7 and Sigma Plot 9.

### 4. Anti-MLH1 Antibodies Detect Only Interfering Crossovers

In plants, immunocytochemical approaches have not yet been followed because of lack of suitable antibodies. However, in tomato a detailed recombinational map has been constructed based on the ultrastructural detection of late RNs along Synaptonemal Complexes (SCs) (Sherman and Stack, 1995, supra).

In order to construct a detailed recombination map of tomato using immunocytology, one has to be able to identify the 12 chromosomes of tomato and the position of recombination nodules (RN). Chromosome identification in tomato is possible based on the relative length of the SCs and their respective arm length ratio.

We have isolated tomato cDNA clones encoding MLH1, cohesins SMC1 and SMC3 and kinetochore protein CENP-C, and prepared antibodies recognizing these proteins, as described above. The anti-SMC1 antibody labels the axial elements of SCs, whereas anti-MLH1 labels intense foci along pachytene SCs. The anti-CENP-C antibody strongly labels the centromeric region on spread preparations of pachytene tomato meiocytes.

Analysis of 113 spread pachytene nuclei were used to construct a detailed recombination map of Cherry tomato and to compare these results with previously published Electron Microscopy data of Sherman and Stack (1995, supra).

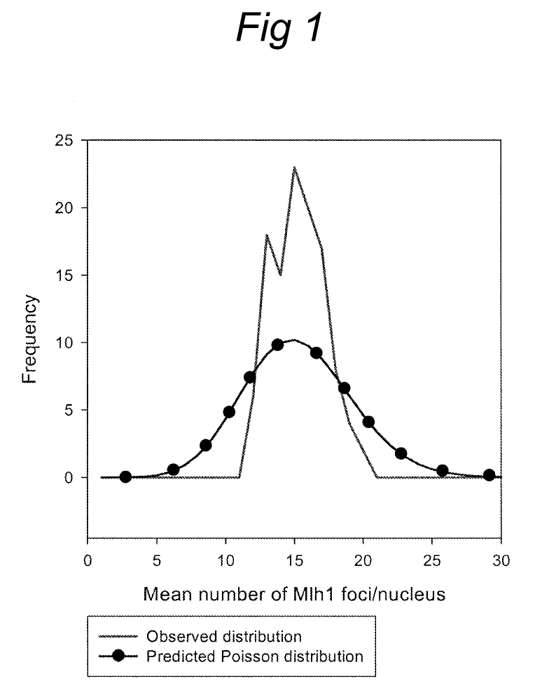

Spread preparation of tomato pollen mother cells were labeled with a rabbit-anti-SMC1 or rabbit-anti-SMC3 antibody, a rabbit-anti-MLH1 antibody and a rabbit-anti-CENP-C antibody. DNA was counterstained with DAPI. The individual immunofluorescent images were overlaid. Images were processed and assembled using Adobe Photoshop 7 and Image J. Results are presented in Table 1 and FIG. 1.

TABLE 1

| | Number of | | | Interference | | | |
|---|---|---|---|---|---|---|---|
| | MLH1 foci or | | | between MLH1 foci | | % SCs without | |
| | late RNs per SC | | | or late RNs | | MLH1 foci | |
| | | | | MLH1 | | | |
| | MLH1 foci | | | foci | late RNs | | |
| SC | (SD) | Late RNs¹⁾ | Difference | \hat{ν} (SE)²⁾ | \hat{ν} (SE)^{1) 2)} | Observed | Expected^{1) 3)} |
| 1 | 1.72 | 2.48 | 0.76 | 9.2 | 2.3 (0.27) | 0.0 | 9.4 |
| | (0.59) | | | (0.97) | | | |
| 2 | 1.33 | 2.08 | 0.75 | 4.8 (1.1) | 2.9 (0.15) | 0.0 | 14.6 |
| | (0.47) | | | | | | |
| 3 | 1.59 | 2.1 | 0.51 | | 2.5 (0.18) | 0.0 | 9.5 |
| | (0.58) | | | | | | |
| 4 | 1.32 | 1.89 | 0.57 | | 3.0 (0.35) | 0.0 | 13.7 |
| | (0.49) | | | | | | |
| 5 | 1.17 | 1.67 | 0.50 | | 2.7 (0.76) | 1.8 | 15.3 |
| | (0.42) | | | | | | |
| 6 | 1.12 | 1.73 | 0.61 | | 3.2 (0.25) | 0.9 | 17.7 |
| | (0.35) | | | | | | |
| 7 | 1.26 | 1.77 | 0.51 | | 2.5 (0.21) | 0.9 | 13.8 |
| | (0.46) | | | | | | |
| 8 | 1.13 | 1.68 | 0.55 | | 3.3 (0.28) | 0.0 | 16.2 |
| | (0.34) | | | | | | |
| 9 | 1.19 | 1.58 | 0.39 | | | 0.9 | 13.2 |
| | (0.42) | | | | | | |
| 10 | 1.16 | 1.66 | 0.50 | | | 1.9 | 15.8 |
| | (0.41) | | | | | | |
| 11 | 1.17 | 1.66 | 0.49 | | | 1.9 | 15.1 |
| | (0.40) | | | | | | |
| 12 | 1.20 | 1.59 | 0.39 | | | 1.8 | 13.4 |
| | (0.44) | | | | | | |

| | | |
|---|---|---|
| sum | 15.36 | 21.89 |

¹⁾data from Sherman and Stack, Genetics 141 (1995) 683-708
²⁾\hat{ν} is the maximum likelihood estimate of the interference parameter ν in the gamma model (with estimated standard error)
³⁾Expected if the difference between the number of late RNs and MLH1 foci were due to random failure of labeling late RNs with anti-MLH1

Table 1 shows the comparison between the amount of MLH1 foci per SC as detected by immunofluorescence (IF) and the amount of late RNs as detected by electron microscopy (EM)(1). The last 2 columns represent the observed percentages of SCs without any MLH1 foci, and the percentages expected if the difference between RNs and MLH1 foci is due to a random failure to detect MLH1 (preparation artifacts), or a limited length of stay of MLH1 in RNs.

The length of spread SCs from 113 tomato nuclei, the position of MLH1 foci and of the centromeres were semi-automatically measured using a homemade macro for the image analysis program Object-Image. Object-Image is a public-domain program developed by N. Vischer at the University of Amsterdam, that is an extended version of NIH Image (developed at the National Institutes of Health by Wayne Rasband). Object. Image is available from http://simon.bio.uva.nl.

Individual SCs were identified based on their relative length and their arm length ratio(1). Table 1 summarizes the results and compares them to these of Sherman and Stack (1995, supra).

Before starting the experiments it was expected that in tomato, the number and positions of the immunocytochemically detected MLH1 foci would correspond with those of the ultrastructurally detected late RNs, and would represent all crossovers. The reasons for expecting this were as follows:

1) The ultrastructurally detected late RNs correspond closely with the genetic map, both with respect to chromosomal position and frequency (Sherman and Stack, 1995). Therefore, late RNs mark most likely all crossovers.

2) MLH1 is essential for meiotic crossing over in all organisms analyzed thus far (reviewed by Hoffmann, E. R. and Borts, R. H. 2004. Meiotic recombination intermediates and mismatch repair proteins. Cytogenetic and Genome Research 107: 232-248.).

3) Accordingly, in the mouse, the positions of MLH1 foci along chromosomes corresponds with crossover positions (Froenicke, L., Anderson, L. K., Wienberg, J., and Ashley, T. 2002. Male mouse recombination maps for each autosome identified by chromosome painting. Am. J. Hum. Genet. 71: 1353-1368.), and elimination of MLH1 eliminates virtually all crossovers in the mouse (Woods, L. M., Hodges, C. A., Baart, E., Baker, S. M., Liskay, M., Hunt, P. A. 1999. Chromosomal influence on meiotic spindle assembly: abnormal meiosis I in female MLH1 mutant mice. J. Cell Biol. 145: 1395-1406).

It was, therefore, completely surprising to find that about 30% less MLH1 foci were detected than late recombination nodules. This 30% difference between MLH1 foci and late RNs is not due to random failure of detecting MLH1 in late RNs, or a limited length of stay of MLH1 in late RNs, because that would have resulted in a much higher frequency of SCs with zero MLH1 foci (Table 1 and Calculations below). The MLH1 foci therefore represent a separate class of RNs, which is placed in such a way onto the chromosomes, that every chromosome gets at least one MLH1 focus.

Another difference between MLH1 foci and late RNs is that MLH1 foci display a much higher level of interference than late RNs. The level of interference can be expressed in the interference parameter n (see Calculations below). For the long arm of chromosome 1, n equals 7 for the MLH1 foci, but only 2.9 for the RNs (Sherman and Stack, 1995). Therefore, the inventors also analyzed whether the distribution of RNs as observed by Sherman et al. (1995) could have resulted from a mixture of high-interference, MLH1-containing RNs and low interference or non-interfering MLH1-negative RNs. Only for the long arm of chromosome 1 we had sufficient observations to test this. The conclusion from this analysis was that the distribution of RNs as observed by Sherman et al. (1995) has most likely arisen by mixing MLH1-containing high-interference RNs with MLH1-negative RNs, and that the MLH1-positive and MLH1 negative RNs are recruited from a common population of precursors that displays a low level of interference. The inventors also tested whether presumed precursors of late RNs (early RNs) display a low level of interference, and this turned out to be the case (data for early RNs taken from Anderson et al., 2001, Genetics 159: 1259-1269).

Since the observed percentage of SCs without MLH1 foci is far lower than expected if the difference between RNs and MLH1 foci were due to random failure to detect MLH1, or to a limited length of stay of MLH1 in RNs (Table 1), this difference has other causes. In Arabidopsis, there are two classes of crossovers; 70-75% are interference-sensitive and the remaining 25-30% are interference-insensitive (Chen et al. 2005). The MLH1 foci display interference and since the anti-MLH1 antibody only detects about 70% of the late RNs as observed by EM, it is concluded that tomato, like Arabidopsis, has two classes of crossovers, one interfering and one non-interfering class of crossovers. In addition, it is concluded that only the RNs that represent interfering crossovers contain MLH1.

In conclusion, anti-MLH1 antibodies detect specifically interfering homologous recombination events. This finding is useful for both quantifying of interfering meiotic homologous recombination events and for determining the positions on chromosomes of those RNs that represent interfering crossovers. Also, a tool is provided to measure effects of various conditions and genetic constitutions on the different pathways.

### Calculations 1

Calculation of expected frequency of SCs with zero MLH1 foci, assuming that 30% of the crossovers/late RNs are not labeled with anti-MLH1 because of random failure of detecting MLH1 in late RNs, or a limited length of stay of MLH1 in late RNs.

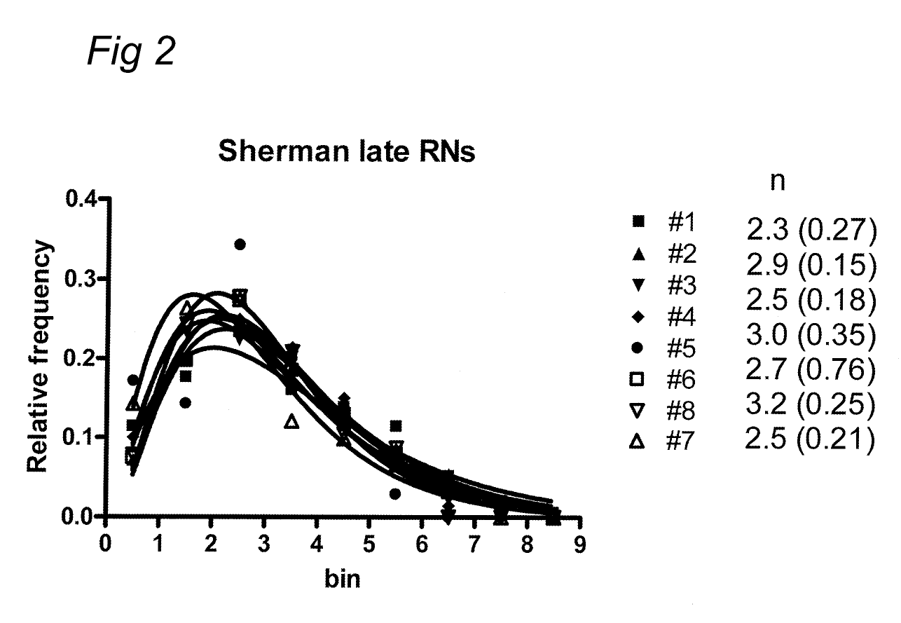

For this calculation we needed to know
the frequency of late RNs; these data are from Sherman and Stack (1995) Table 5.
how the late RNs are distributed along each chromosome. For this we analyzed the data in Table 10 of Sherman and Stack, which shows the position of late RNs in the euchromatin of the long arms of the 12 tomato chromosomes. For chromosomes 1 to 8 there were sufficient observations to analyze how the RNs were distributed. For all analyzed chromosomes we found that the distances between adjacent RNs could be fitted to a gamma distribution (explanation see below), with estimations of the interference parameter n between 2.3 and 3.2, see FIG. 2.

In FIG. 2 the inter-RN distances are plotted against the relative frequencies of those distances; the symbols represent the observations in Table 10 from Sherman and Stack, and the lines show the best fits to the gamma distribution. If the inter-RN distances can be fitted to a gamma distribution, this might mean that there is interference between RNs. n is a parameter in the formula for the gamma distribution. The table to the right shows for which n-values we obtained the best fits. If the RNs were distributed randomly along the chromosomes, n would equal 1; if n>1, then there is interference between RNs, and the higher n is, the stronger interference is. For further calculations we assumed that late RNs along tomato chromosomes display a low level of interference, with n=3.

Then we calculated the expected frequency of SCs with zero MLH1 foci, if the immunocytochemical detection of MLH1 would fail at random in 30% of the late RNs.

For each chromosome, we simulated on at least 5000 copies the positions of late RNs, given the average frequency of late RNs on that chromosome (Sherman Table 5), and assuming n=3. Subsequently we took away at random the fraction of RNs that was not labeled by anti-MLH1 (for instance, for chromosome 1 this fraction was 0.76/2.48=0.31), and determined the percentage of SCs of that chromosome that would have zero MLH1 foci. This percentage is much higher than what we observed (Table 1), and we conclude therefore that the difference between the frequency of MLH1 foci and late RNs is not due to random failure to detect MLH1 in late RNs, and that the MLH1 foci represent a separate class of RNs (and thus of crossovers).

### Calculations 2

The questions we tried to answer were: Is it possible to obtain a distribution of late RNs as observed by Sherman and Stack (1995) by mixing high-interference MLH1-positive RNs with MLH1-negative RNs? And if so, how are the MLH1-negative RNs distributed along SCs?

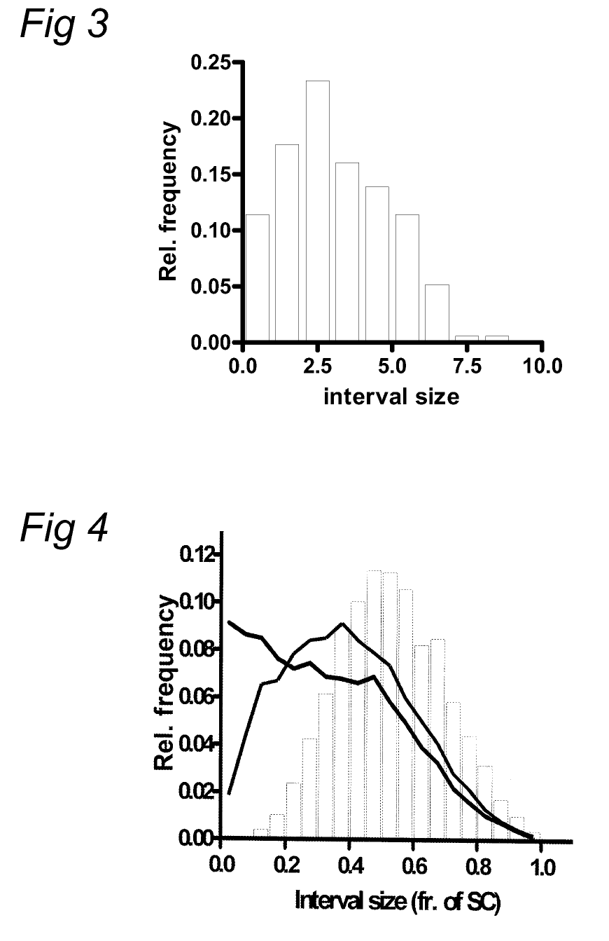

FIG. 3 shows the frequency distribution of distances between late RNs (interval sizes) on the long arm of Chromosome #1. The bars represent data from Sherman and Stack, 1995, Table 10, (the line represents the best fit to the gamma distribution; this fit was obtained for n=2.9). Interval sizes are given in arbitrary units.

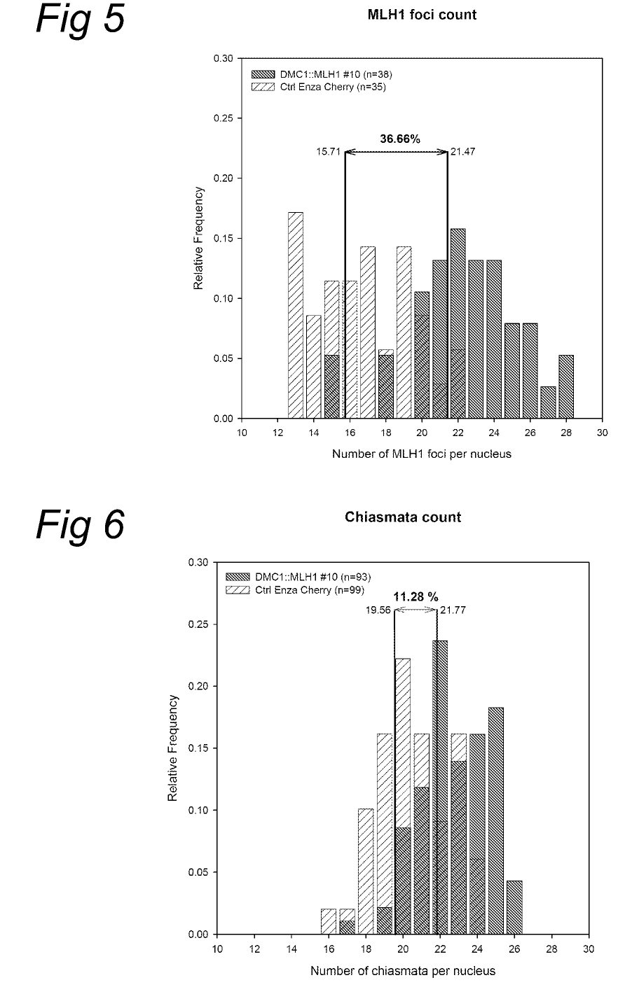

FIG. 4 shows the expected frequency distribution of inter-RN distances. The horizontal axis is drawn to scale with that of FIG. 3.

The bars represent the expected distribution of distances between MLH1 foci only, for 1.4 foci/long arm and n=7. This distribution is similar to the inventors' observations, and differs clearly from that in FIG. 3: the peak is further to the right, and there are no very small interfocus distances.

In simulations we found that if the interference-negative RNs are placed entirely independently of the MLH1 foci, there will always be more small intervals than observed by Sherman and Stack. An example is shown by the brown/dark colored line, which represents the expected distribution of distances between RNs (MLH1 positive and negative), if on average 0.63 non-interfering, MLH1-negative RNs were placed per long arm of chromosome 1 in addition to on average 1.4 MLH1 foci per long arm, in such a way that the MLH1-positive RNs do not influence the position of MLH1 negative RNs and vice versa. This would yield far more small interfocus distances than Sherman and Stack found (Sherman and Stack, 1995).

However, if we assume that the MLH1 positive, high-interference RNs and the MLH1-negative RNs are not placed entirely independently, but originate from the same population of precursors, and that these precursors display already a low level of interference (n=2), we get a similar distribution of inter-RN distances as obtained by Sherman and Stack (yellow/light colored line).

In short, we conclude from these simulations that MLH1 marks a subpopulation of strongly interfering RNs (and thus crossovers), and we propose that the MLH1-positive and MLH1-negative RNs originate from the same population of precursors that display already a low level of interference.

We tested one aspect of this model, namely that precursors of late RNs already display a low level of interference: Early RNs are candidate precursors of late RNs, and we found that they display a low level of interference (n=2-3) (data taken from Anderson et al., 2001, supra).

Another testable aspect of the model is that 70% of the ultrastructurally detectable late RNs contain MLH1. This will be tested by immuno-EM, using anti-MLH1 antibodies.

### 5. Lemlh1 Sequence Optimization for Expression in Tomato

In the fully sequenced ORF of the LeMLH1 gene, many recognition sites of commonly used restriction enzymes are present.

Without changing the amino acid translation, the following sites have been removed (ATG on position 252)): NcoI at position 251, EcoRI at position 604, HindIII at position 1004 and SacI at position 156 and 1356. The NcoI site has been removed so that a new NcoI site can be used for future cloning. The EcoRI site has been removed to be able to use the EcoRI site at the end of the nos terminator. The HindIII site has been removed to make it possible to use the native HindIII site near the end of the AtDMC1 promoter. The SacI site has been removed for the use of a similar site at the 5′ end of the nos terminator.

A part of the LeMLH1 3′ UTR has been maintained. The length of the sequence between the stop codon of the LeMLHI ORF and the polyA signals in the nos terminator has been kept identical to the one in the gusA expression construct. Due to the sequence optimization (see below) the BamHI site at position 587 has been disappeared, while a new BamHI site appeared at position 1223. Subsequently, the gusA sequence was replaced by the optimized Lemlh1 sequence below.

The Lemlh1 nucleic acid sequence was optimized as follows:

The codon usage was adapted to the codon bias of Lycopersicon esculentum (tomato) genes, while regions of very high (>80%) or very low (<30%) GC content have been avoided where possible. This was done by GeneArt (Germany). The LeMLH1 ORF was optimized with the goal to reach a relative high GC content of about 50%. In nature the GC content is normally between 30 to 40%, but a higher content is expected to enhance transgenic expression. Many nucleotides in the ORF have thus been changed, but the predicted amino acid sequence after translation has been kept the same.

Furthermore, during the codon optimization processes, the following cis-acting sequence motifs were avoided:
internal TATA-boxes, chi-sites and ribosomal entry sites;
AT-rich or GC-rich sequence stretches;
RNA instability elements;
repeat sequences and RNA secondary structures;
(cryptic) splice donor and acceptor sites.

The optimized Lemlh1 nucleic acid sequence, which encodes the same amino acid sequence as the wild type cDNA, is shown in SEQ ID NO: 2 and was used to construct an expression vector (below).

### 6. Vector Construction and Production of Transgenic Tomato Plants

The aim of this task is to clone LeMLH1 behind the ATDMC1 promoter. Following a ‘classical’ recombinant cloning strategy, new adapters need to be ligated to LeMLH1 and the ATDMC1 promoter to facilitate directional cloning. However, after further analysis of the sequences it appeared to be very difficult to accomplish such strategy, because the promoter and LeMLH1 contain many restriction sites. As a result, a lot of different cloning steps are needed to generate the final ATDMC1::LeMLH1 construct using the ‘classical’ cloning method (if possible at all).

It was decided to choose an alternative approach which is to design the optimal AtDMC1::LeMLH1 construct in silico, and order the artificial synthesis of LeMLH1 with some flanking sequence. As a result, the ATDMC1::LeMLH1 construct was made of which the promoter or the coding sequence can easily be exchanged by another sequence.

The sequence to synthesize contains: the end part of the ATDMC1 promoter, the 5′ UTR of gus, LeMLH1 coding sequence, the 3′ UTR of LeMLH1, the nos terminator, and additional restriction sites.

Tomato plants were transformed with the construct and transgenic plants were regenerated.

Cytological assays were carried out on these plants.

### 7. Increased Meiotic Homologous Recombination (MHR) in Tomato Plant Overexpressing the MLH1 Gene (DMC1::MLH1)

### Methods

Plant material: Control plants were Enza cherry plants. Transgenic plants were DMC1::MLH1 transformant no. 10.

### Chromosome Spread Preparation for Immunofluorescence

### Digestion Medium (TE)

Use 800 mM Tris-HCl, 500 mM EDTA pH 7.01 100X Stock. To prepare, weigh the appropriate amount of Tris and EDTA, dissolve them in MilliQ water, adjust pH to 7 with HCl complete to final volume with MilliQ. Add 0.7 M mannitol (Sigma, MW 182.17, 637.7 mg/5 mL) and 1% PVP (44,000 in MW, 50 mg/5 mL). Dissolve completely and use as is (pH 6.98). To 1 mL of digestion medium add 5 mg of freeze-dried desalted cytohelicase from Helix pomatia (Sigma C-8274), 3 mg of cellulase onozuka (Yakult Honsha Co LTD) and 3 mg of pectolyase from Aspergillus japonicus (Sigma P-3026). Mix gently to fully dissolve the enzymes.

### Bursting Medium

### 0.5% NP40

### 0.1% BSA

### Freshly-Prepared 2% PFA Solution

Mix 1 g PFA with 50 mL MilliQ. Add 1 drop of 1N NaOH. Warm up to 60° C. until complete dissolution. Cool down on ice. Filter on 0.2 μm filter.

Keep solutions on ice.

### Glow-Discharged Slides

Sonicate slides from 15 minutes in 70% ethanol. Boil the slides in MilliQ water for 20-30 minutes boiling time. Place the hot glass slides on a tube rack and allow them to air-dry overnight. Place the clean glass slides in a plastic tray. Glow-discharge the slides for 5 min. at 3 A with a vacuum of 0.1 torr (adjust the vacuum with argon).

### Spreading Procedure

Dissect the flower bud under the dissecting microscope. Collect one anther, measure the length (2.1 mm for pachytene) and cut the tip on a clean glass slide. Squeeze the PMCs out of the anther and apply immediately one droplet of a 2% aceto-orcein solution. Cover with a coverslip. Flame for a few seconds using an alcohol lamp. Apply a paper tissue over the coverslip and tap the coverslip with the blunt end of a dissecting needle. Flame the slide on an alcohol lamp and observe with phase contrast. If the staging is right, collect the 4 remaining anthers and transfer them to a depression slide containing 200 μL of digestion medium. Cut the anther tips and squeeze gently the PMCs out in the digestion medium (if PMCs disperse in the digestion medium, they are probably in later stages than pachytene, extend the digestion time until the PMCs are completely translucent). Incubate in the digestion medium for 20 min. at 25° C. in a wet incubator. Using a siliconized micro-pipette, collect up to 4 rods of cells in a volume of digestion medium as little as possible (at most 0.5 μL). Expel the cells in a 10 μL droplet of bursting medium hanging at the tip of a micro-pipette.

Apply the droplet of spermatocyte lysis buffer containing the cells on a 10 μL droplet of freshly-prepared 2% PFA (Final PFA concentration on slide 1%) placed in the centre of a glow-discharged slide. Allow the cells to swell and burst in a tightly closed wet chamber for approximately 1 hour to 90 min. Allow the slides to completely air-dry Check if the spreads are OK with phase contrast optics. Wash the slides 5 min. in 0.4% Photoflo 200 in water. Wash the slides 1 times 5 min. in MilliQ water. Even slides are washed in PBS and immediately processed for Immunolabeling.

### Immunolabeling

Even slides are blocked for 30 min. at RT in 1% BSA, 0.1% Triton X100, 0.05% NaN3 in filtered PBS pH 7.4. Slides are incubated 1 hr at 37° C., 48 hrs at 4° C. and 1 hr at 37° C. in a wet chamber in the dark with 100 μL/slide of a RabαLeMLH1 diluted 1:100 in blocking and centrifuged for 30 min. at 4° C. Slides are washed 3 times 5 min. in filtered PBS. Slides are incubated 2 hrs at 37° C. in a dark wet chamber with 100 μL/slide of a GαRab-FITC-Fab diluted 1:200 in blocking. Slides are washed 3 times in filtered PBS in the dark. Slides are incubated 1 hr at 37° C., o/n at 4° C. and 1 hr at 37° C. in a dark wet chamber with 100 μL/slide of a RαLeSMC1 diluted 1:50 in blocking and centrifuged for 30 min. at 4° C. Slides are washed 3 times in filtered PBS. Slides are incubated 2 hrs at 37° C. in a dark wet chamber with 100 μL/slide of a GαRab-TR diluted 1:200 in blocking. Slides are washed 3 times in filtered PBS. Slides are mounted in Vectashield containing 1 μg/mL DAPI and sealed with clear nail polish. Slides are stored at −20° C. until observation (about 4 days at −20° C.).

Chromosome Spreads Preparation for Chiasmata Count [Zhong et al. Chromosome Research 4:24-28 (1996)]

Buds from control Enza Cherry tomatoes and DMC1::MLH1 transformant no. 10 tomatoes were collected at different stages and stored in tubes containing moist paper. Buds were dissected and diakinesis and late diplotene stages were identified by mean of squashes in 2% aceto-orcein.

The remaining 4 anthers are fixed for 20 min. in Carnoy's fluid (ethanol:acetic acid 3:1).

Anthers are washed twice with distilled water and digested for 2 hrs at 37° C. in 2 mL of 0.3% pectolyase, 0.3% cellulase, 0.3% cytohelicase in 30 mM sodium citrate buffer adjusted to pH 4.5 with 1N HCl.

Anthers are washed in distilled water 3 times and stored on ice until use.

Transfer a single anther to a clean glass slide.

Add 5 μL of distilled water, cut the anther tip and squeeze the PMCs out of the anther sacs.

Add 50 μL (10 vol.) of 50% acetic acid to the cell suspension, transfer the slide to a hot plate (42° C.) and mix using a thin needle without touching the glass slide (drag the droplet around with the needle) for appr. 60 sec.

During this step the cytoplasm should lyse and the acetic acid evaporate.

Add 1 mL of ice-cold Carnoys fixative around the droplet of water containing the cells and still using the needle drag the water in circles until it mixes with the Carnoy's fixative.

Just prior to complete evaporation, add some more fixative and let dry.

Immerse the slide briefly into 96% ethanol and dry with a hair drier.

The slides can be stored at −20° C. for months.

The slides are immediately stained with 10 μg/μL DAPI in Vectashield and stored at −20° C. until observation.

### Results

38 and 93 nuclei from the DMC1::MLH1 transformant no. 10, and 35 and 99 nuclei from control plants, were analyzed by immunofluorescence and chiasmata count, respectively. Relative frequency distribution plots were generated and populations were compared statistically by mean of unpaired t-test. Results are shown in FIGS. 5 and 6.

Immunofluorescence data revealed the average number of MLH1 foci in the MLH1 overexpressing plant no. 10 is significantly higher than that of the control plants (21.47 vs. 15.71 respectively, p<0.001), which represents a net increase of 36.67% (FIG. 5). Chiasmata count data revealed that the average number of chiasmata in the MLH1 overexpressing plant no. 10 is significantly higher than that of the control plants (21.77 vs. 19.56 respectively, p<0.001), which represents a net increase of 11.28% (FIG. 6).

1. A method for producing a transgenic plant cell or plant having an altered ratio of interfering to non-interfering meiotic homologous recombination events and/or an altered positioning of meiotic homologous recombination events on its chromosomes, compared to a non-transgenic plant, said method comprising the step of:
transforming a plant or plant cell with a nucleic acid molecule encoding an active MLH1 protein the amino acid sequence of which is at least 70% identical to SEQ ID NO:3, operably linked to a promoter active in plant cells,
wherein said altered ratio is determined by contacting an anti-MLH1 antibody with meiotic chromosome spreads of said plants and wherein said altered positioning is determined in a cytological assay using an anti-MLH1 antibody, an antibody that detects the axial elements of synaptonemal complexes and an antibody that labels the centromere region.

2. The method according to claim 1, wherein said nucleotide molecule is integrated into the plant's genome.

3. The method according to claim 22, further comprising step of producing another plant or a population of plants from said regenerated plant and, optionally, selecting one or more plants from said population.

4. The method according to claim 1 wherein the total frequency of meiotic homologous recombination is not significantly altered from the frequency in a corresponding non-transgenic plant.

5. (canceled)

6. The method according to claim 1, wherein the promoter is a meiosis-preferred or a meiosis-specific promoter or an inducible promoter.

7. The method according to claim 1, wherein codon usage of the nucleotide molecule is the codon usage of the genus or species of the transformed plant cell or plant.

8. The method according to claim 1, wherein the cytological assay comprises the steps of;
(a) preparing a specimen of meiotic pachytene cells,
(b) contacting said specimen with at least an anti-MLH1 antibody, and
(c) determining the number of labeled MLH1-foci per cell.

9. The method according to claim 1, wherein the plant is a member of the family Solanaceae.

10. A transformed plant, plant seed, plant cell, or a population of transformed plants, seeds or cells, obtainable by the method according to claim 1.

11. A method for determining the frequency of interfering meiotic homologous recombination events in plant cells, comprising:
(a) contacting a specimen of meiotic pachytene plant cells with an anti-MLH1 antibody, and
(b) determining the number of labeled MLH1-foci per cell.

12. A method for determining the location and distribution of interfering meiotic homologous recombination events in a plant cell, comprising:
(a) contacting a specimen of meiotic pachytene plant cells simultaneously or sequentially with an anti-MLH1 antibody, an antibody that labels the axial elements of the synaptonemal complex and an antibody that labels the centromere region, and optionally, counterstaining chromosomal DNA with DAPI, and
(b) determining the number of labeled MLH1-foci per cell.

13. The method according to claim 25, wherein the antibody that labels the axial elements of the synaptonemal complex is an anti-SMC1 or an anti-SMC3 antibody and the antibody that labels the centromere region is an anti-CENP-C antibody.

14. The method according to claim 11, wherein the anti-MLH1 antibody is specific for an epitope comprising
(i) at least 5 consecutive amino acids of SEQ ID NO:3, or
(ii) at least 5 consecutive amino acids of a sequence that is at least 50% identical with SEQ ID NO:3.

15. The method according to claim 13, wherein said anti-MLH1 antibody, said anti-SMC1 antibody, said anti-SMC3 antibody and said anti-CENP-C antibody are specific for SEQ ID NO:4, SEQ ID NO 6, SEQ ID NO:10 and SEQ ID NO:8, respectively, or against a fragment of these sequences that comprises at least 5 consecutive amino acids of said sequences.

16. A method for detecting meiotic homologous recombination events in plant cells or alteration of the frequency and/or positioning of meiotic homologous recombination events in plants using an MLH1 protein, a nucleic acid encoding the MLH1 protein, or an antibody specific for the MLH1 protein.

17. A polyclonal or monoclonal antibody raised against amino acid sequence SEQ ID NO:4, or against a fragment thereof, comprising at least 5 consecutive amino acids of SEQ ID NO:4.

18. An isolated polypeptide comprising the amino acid sequence SEQ ID NO:3, or comprising an amino acid sequence that is at least 60% identical with SEQ ID NO:3.

19. An isolated nucleic acid molecule encoding the isolated polypeptide according to claim 18.

20. The nucleic acid molecule according to claim 19, the sequence of which comprises SEQ ID NO:1 or SEQ ID NO:2.

21. An isolated nucleic acid molecule encoding an MLH1 protein, characterized in that:
(a) the GC content of said nucleic acid molecule has been modified to be the same or higher than the GC content of a nucleic acid encoding an MLH1 protein of a species from which said nucleic acid molecule was isolated,
(b) the modified GC content does not alter the amino acid sequence of said encoded MLH1 protein, and/or
(c) restriction enzyme recognition sites for at least 2 different restriction enzymes have been removed from said nucleic acid molecule without altering the amino acid sequence of said encoded MLH1 protein.

22. A method for producing the transgenic plant of claim 1, further comprising the step of regenerating a plant from said plant or cell transformed in accordance with claim 1.

23. The method according to claim 9 wherein the plant is a member of the genus Solanum.

24. The method of claim 11 wherein said determining step (b) employs light or electron microscopy.

25. The method of claim 11 wherein the specimen is further contacted in step (a) with one or more of:
(i) an antibody that binds to and labels the axial elements of the synaptonemal complex; and
(ii) an antibody that binds to and labels the centromere region.

26. The method of claim 11 further comprising counterstaining chromosomal DNA with DAPI

27. The method of claim 25 further comprising counterstaining chromosomal DNA with DAPI

28. The method of claim 12 wherein step (a) comprises the counterstaining of chromosomal DNA with DAPI.

29. The method of claim 12 wherein said determining step (b) employs light or electron microscopy.
